(19) United States
(12) Patent Application Publication
(10) Pub. No.: US 2017/0042944 A1
(43) Pub. Date: Feb. 16, 2017

(54) TREATMENT OF CONDITIONS AND COMPLICATIONS IN INFANTS

(71) Applicants: Robert HARIRI, San Diego, CA (US); Jodi P. GURNEY, San Diego, CA (US); Xiaokui ZHANG, San Diego, CA (US); Stacy HERB, San Diego, CA (US); Cindy HARTUNG, San Diego, CA (US); ANTHROGENESIS CORPORATION, Warren, NJ (US)

(72) Inventors: Robert J. HARIRI, Bernardsville, NJ (US); Jodi P. GURNEY, Chicago, IL (US); Xiaokui ZHANG, Livingston, NJ (US); Stacy Herb, Mountain Top, PA (US); Cindy HARTUNG, Durham, NC (US)

(73) Assignee: ANTHROGENESIS CORPORATION, Warren, NJ (US)

(21) Appl. No.: 15/305,325

(22) PCT Filed: Apr. 20, 2015
(86) PCT No.: PCT/US15/26610

§ 371 Date: Oct. 19, 2016

Related U.S. Application Data
(60) Provisional application No. 61/982,144, filed on Apr. 21, 2014.

(51) Int. Cl. A61K 35/50; A61K 9/00; A61K 35/51

(52) U.S. Cl. CPC A61K 35/50; A61K 35/51; A61K 9/0019; A61K 9/0085

(57) ABSTRACT

Provided herein are compositions and methods for treating one or more disorders or conditions in infants, including premature infants, by administering to such infants umbilical cord blood or cells obtained from umbilical cord blood and, optionally, cells obtained from placental perfusate, placental stem cells and/or blood additives. Also provided herein are methods for treating one or more disorders or conditions in infants, including premature infants, by administering to such infants cells obtained from placental perfusate alone or placental stem cells alone or in combination with umbilical cord blood or cells obtained from umbilical cord blood.

This application claims benefit of U.S. Provisional Patent Application No. 61/982,144, filed Apr. 21, 2014, the disclosure of which is incorporated by reference herein in its entirety.

### 1. FIELD

Provided herein are compositions and methods for treating one or more disorders or conditions in infants, including premature infants, by administering to such infants umbilical cord blood or cells obtained from umbilical cord blood and, optionally, cells obtained from placental perfusate, placental stem cells and/or blood additives. Also provided herein are methods for treating one or more disorders or conditions in infants, including premature infants, by administering to such infants cells obtained from placental perfusate alone or placental stem cells alone or in combination with umbilical cord blood or cells obtained from umbilical cord blood.

### 2. BACKGROUND

A full term infant spends 37 to 42 weeks in the uterus. An infant born earlier than 37 weeks is considered premature or preterm. Premature birth is the leading cause of death in the first month of life and is a major public concern. According to the March of Dimes Birth Defects Foundation, in 2002 there were 480,812 premature births (representing 12.1% of all live births) in the United States, and the cost for medical care of premature infants was 15.5 billion dollars.

Risk factors for preterm labor and delivery include age of the mother (less than 18 years of age or greater than 35 years of age), infection, diabetes mellitus, hypertension, smoking, a pregnancy with multiple fetuses and substance abuse.

Currently, survival rates for infants born from about 23 to about 25 weeks of gestation vary from about 20 percent for infants of 23 gestational weeks, to about 65 percent for infants of 25 gestational weeks. About one third of surviving babies in this age group develop normally; about one third develop mild or moderate disabilities; and about one third develop severe disabilities.

Survival rates for premature infants born from about 26 to about 29 weeks of gestation are about 75 percent for infants born at 26 gestation weeks and about 85 percent for infants born at 29 gestation weeks. About 40 percent of such premature infants who survive develop normally, while about 40 percent develop mild or moderate disabilities and about 20 percent develop one or more severe disabilities.

90 to 95 percent of premature infants born from about 30 to about 33 weeks of gestation survive. About 65% of these premature infants develop normally, while about 20 percent develop mild or moderate disabilities and about 15 percent of these premature infants develop one or more severe disabilities.

Although premature infants born from about 34 to about 37 weeks of gestation are less mature than full-term infants, their survival rate (around 95 percent) is nearly identical to the survival rate for full-term infants, and their long-term prospects are as good as those of any full-term infant.

Physical features of a premature infant include, for example, small size; low birth weight; irregular breathing; and underdeveloped organs or systems such as lung, immune system and brain. Common problems associated with premature infants include but are not limited to anemia, low blood pressures, hyperbilirubinemia, infection, retinopathy, respiratory distress and incomplete development of certain organs, such as lung, eye, immune system, brain, heart, liver and kidney.

Various treatment options, having varying degrees of success, are available to treat disorders and conditions associated with infants, including premature infants. For example, premature infants with anemia may be treated with blood transfusions and/or iron supplementation, and surfactant administration is used to treat respiratory distress syndrome associated with preterm birth. No treatment options exist, however, for incomplete organ development. Despite progress, a need continues to exist for development of treatments for disorders and conditions associated with premature infants.

### 3. SUMMARY

Provided herein are methods of treating disorders and conditions of infants (e.g., full-term infants and premature infants), including disorders and conditions of premature infants caused by or associated with premature birth.

In one aspect, provided herein is a method of treating a disorder or condition of a premature infant.

In a specific embodiment, provided herein is a method of treating a disorder or condition of a premature infant, wherein the disorder or condition is caused by or associated with premature birth, comprising administering to the premature infant a composition comprising umbilical cord blood (e.g., allogeneic cord blood or autologous cord blood), or cells obtained therefrom (e.g., total nucleated cells from umbilical cord blood). In specific embodiments, the method additionally comprises administering to the premature infant cells obtained from placental perfusate (e.g., total nucleated cells from placental perfusate), placental stem cells and/or blood additives. In embodiments in which cells obtained from placental perfusate (e.g., total nucleated cells from placental perfusate), placental stem cells and/or blood additives are administered with umbilical cord blood or cells obtained therefrom (e.g., total nucleated cells from umbilical cord blood), the cells obtained from placental perfusate or placental stem cells can be, e.g., autologous or allogeneic cells obtained from placental perfusate or placental stem cells and, likewise, the umbilical cord blood or cells obtained therefrom can be, e.g., autologous or allogeneic umbilical cord blood. In a more specific embodiment, said blood additive is erythropoietin, an iron supplement, a vitamin, or allogeneic red blood cells not obtained from cord blood. In a more specific embodiment, wherein said additive is allogeneic red blood cells, said cells are irradiated to a degree sufficient to reduce or prevent graft versus host disease. In a specific embodiment, said irradiation is with at least 2,500 cGy of radiation. In another more specific embodiment, wherein said additive is allogeneic red blood cells, said cells have been leukodepleted.

In another specific embodiment, provided herein is a method of treating a disorder or condition of a premature infant, comprising administering to the premature infant a composition comprising cells obtained from placental perfusate (e.g., total nucleated cells from placental perfusate). In a specific embodiment, the placental perfusate is autologous, i.e., the placental perfusate is derived from the placenta of the premature infant being treated.

In another specific embodiment, provided herein is a method of treating a disorder or condition of a premature infant, comprising administering to the premature infant a composition comprising placental stem cells. In a specific embodiment, the placental stem cells are autologous placental stem cells, i.e., the placental stem cells are derived from the placenta of the premature infant being treated. In another specific embodiment, the placental stem cells administered are contained within, or are derived from, placental perfusate.

In another specific embodiment, provided herein is a method of treating a disorder or condition of a premature infant, comprising administering to the premature infant a composition comprising umbilical cord blood and cells obtained from placental perfusate (e.g., total nucleated cells from placental perfusate). In a specific embodiment, provided herein is a method of treating a disorder or condition of a premature infant, comprising administering to the premature infant a composition comprising cells obtained from umbilical cord blood (e.g., total nucleated cells from umbilical cord blood) and cells obtained from placental perfusate (e.g., total nucleated cells from placental perfusate). In embodiments in which cells obtained from placental perfusate (e.g., total nucleated cells from placental perfusate) and umbilical cord blood or cells obtained therefrom are administered, the placental perfusate can be, e.g., autologous or allogeneic placental perfusate and, likewise, the umbilical cord blood can be, e.g., autologous or allogeneic umbilical cord blood. In a specific embodiment, either the cord blood or the placental perfusate are autologous to the infant. In another specific embodiment, both the cord blood and placental perfusate are autologous to the infant.

In another specific embodiment, provided herein is a method of treating a disorder or condition of a premature infant, comprising administering to the premature infant a composition comprising umbilical cord blood and placental stem cells. In a specific embodiment, provided herein is a method of treating a disorder or condition of a premature infant, comprising administering to the premature infant a composition comprising cells obtained from umbilical cord blood (e.g., total nucleated cells from umbilical cord blood) and placental stem cells. In embodiments in which placental stem cells and umbilical cord blood or cells obtained therefrom are administered, the placental stem cells can be, e.g., autologous or allogeneic placental stem cells and, likewise, the umbilical cord blood can be, e.g., autologous or allogeneic umbilical cord blood. In a specific embodiment, either the cord blood or the placental stem cells are autologous to the infant. In another specific embodiment, both the cord blood and the placental stem cells are autologous to the infant. In certain embodiments, the placental stem cells administered are placental stem cells contained within, or derived from, placental perfusate.

A premature infant is an infant who is born less than 37 weeks of gestation. In certain embodiments, the premature infant has undergone from about 23 to about 25 weeks of gestation at birth. In certain embodiments, the premature infant has under undergone from about 26 to about 29 weeks of gestation at birth. In certain embodiments, the premature infant has under undergone from about 30 to about 33 weeks of gestation at birth. In certain embodiments, the premature infant has under undergone from about 34 to about 37 weeks of gestation at birth.

In certain embodiments, the premature infant weighs about 800 grams or more at birth. In certain embodiments, the premature infant weighs about 500 grams to about 800 grams at birth. In certain embodiments, the premature infant weighs less than about 500 grams at birth.

In certain embodiments, the premature infant is a neonatal infant, e.g., an infant in its first month of life.

In another aspect, provided herein are methods of treating full term infants (i.e., infants that spent 37 to 42 weeks in the uterus of their mother).

In a specific embodiment, provided herein is a method of treating a disorder or condition of a full term infant, comprising administering to the infant a composition comprising umbilical cord blood (e.g., allogeneic cord blood or autologous cord blood) or cells obtained therefrom (e.g., total nucleated cells from umbilical cord blood). In specific embodiments, the method additionally comprises administering to the infant cells obtained from placental perfusate (e.g., total nucleated cells from placental perfusate), placental stem cells and/or blood additives such as erythropoietin, an iron supplement, a vitamin, or allogeneic red blood cells not obtained from cord blood.

In another specific embodiment, provided herein is a method of treating a disorder or condition of a full term infant, comprising administering to the infant a composition comprising cells obtained from placental perfusate (e.g., total nucleated cells from placental perfusate). In a specific embodiment, the placental perfusate (e.g., total nucleated cells from placental perfusate) is autologous, i.e., the placental perfusate is derived from the placenta of the infant being treated.

In another specific embodiment, provided herein is a method of treating a disorder or condition of a full term infant, comprising administering to the infant a composition comprising placental stem cells. In a specific embodiment, the placental stem cells are autologous placental stem cells, i.e., the placental stem cells are derived from the placenta of the infant being treated. In another specific embodiment, the placental stem cells administered are contained within, or are derived from, placental perfusate.

In another specific embodiment, provided herein is a method of treating a disorder or condition of a full term infant, comprising administering to the infant a composition comprising umbilical cord blood and cells obtained from placental perfusate (e.g., total nucleated cells from placental perfusate). In a specific embodiment, provided herein is a method of treating a disorder or condition of a full term infant, comprising administering to the infant a composition comprising cells obtained from umbilical cord blood (e.g., total nucleated cells from umbilical cord blood) and cells obtained from placental perfusate (e.g., total nucleated cells from placental perfusate). In embodiments in which cells obtained from placental perfusate (e.g., total nucleated cells from placental perfusate) and umbilical cord blood or cells obtained therefrom are administered, the placental perfusate can be, e.g., autologous or allogeneic placental perfusate and, likewise, the umbilical cord blood can be, e.g., autologous or allogeneic umbilical cord blood. In a specific embodiment, both the cord blood and the placental perfusate are autologous to the infant.

In another specific embodiment, provided herein is a method of treating a disorder or condition of a full term infant, comprising administering to the infant a composition comprising umbilical cord blood and placental stem cells. In a specific embodiment, provided herein is a method of treating a disorder or condition of a full term infant, comprising administering to the infant a composition comprising cells obtained from umbilical cord blood (e.g., total nucleated cells from umbilical cord blood) and placental stem cells. In embodiments in which placental stem cells and umbilical cord blood or cells obtained therefrom are administered, the placental stem cells can be, e.g., autologous or allogeneic placental stem cells and, likewise, the umbilical cord blood can be, e.g., autologous or allogeneic umbilical cord blood. In a specific embodiment, both the cord blood and the placental stem cells are autologous to the infant. In certain embodiments, the placental stem cells administered are placental stem cells contained within, or derived from, placental perfusate.

In certain embodiments, the full-term infant is a neonatal infant, e.g., an infant in its first month of life.

The disorder or condition to be treated according to the methods provided herein can be any disorder or condition known in the art to be caused by or associated with premature birth and/or known to afflict infants (e.g., premature infants and full-term infants, including neonatal infants born prematurely and at full-term). In certain embodiments, the disorder or condition is Respiratory Distress Syndrome (RDS) or Acute Respiratory Distress Syndrome (ARDS). In certain embodiments, the disorder or condition is anemia. In certain embodiments, the disorder or condition is intraventricular hemorrhage, necrotizing enterocolitis, retinopathy of prematurity, chronic lung disease (bronchopulmonary dysplasia), an infection, patent ductus arteriosus, apnea, low blood pressure, or hyperbilirubinemia. In certain embodiments, the disorder or condition is caused by incomplete development of an organ, such as lung, eye, immune system, brain, heart, liver or kidney.

In certain embodiments, the disease or disorder treated in accordance with the methods described herein is a neurological disorder. Neurological disorders that can be treated using the methods described herein include, without limitation, intraventricular hemorrhage, hypoxic ischemic encephalopathy, retinopathy of prematurity, cerebral palsy, and white matter brain abnormalities. In a specific embodiment, the methods described herein are used to treat an infant with a neurological disorder, wherein said neurological disorder is caused by ischemia.

In certain embodiments, the disease or disorder treated in accordance with the methods described herein is a gastrointestinal disorder. Gastrointestinal disorders that can be treated using the methods described herein include, without limitation, necrotizing enterocolitis and gastroesophageal reflux disease (GERD).

In certain embodiments, the disease or disorder treated in accordance with the methods described herein is a respiratory disorder. Respiratory disorders that can be treated using the methods described herein include, without limitation, respiratory distress syndrome, apnea (e.g., apnea of prematurity), and bronchopulmonary dysplasia.

Cells obtained from umbilical cord blood (e.g., total nucleated cells from umbilical cord blood) used herein can be isolated from umbilical cord blood by any technique known in the art, and umbilical cord blood used herein can be collected by any technique known in the art. In certain embodiments, umbilical cord blood is obtained from a cord blood bank. In certain embodiments, umbilical cord blood is collected from a pre-term mammalian placenta. In certain embodiments, umbilical cord blood is collected from a post-partum mammalian placenta. In some embodiments, umbilical cord blood is obtained from a post-partum mammalian placenta of a full-term birth. In other embodiments, umbilical cord blood is obtained from a post-partum mammalian placenta of a premature birth. The umbilical cord blood can be allogeneic to the infant (e.g., premature infant or full-term infant) to be treated, if administered alone, or can be allogeneic or autologous, or a combination of both, if administered with placental stem cells or blood additives. In a specific embodiment, the umbilical cord blood used in the methods described herein is autologous to the infant being treated.

Cells obtained from placental perfusate (e.g., total nucleated cells from placental perfusate) used herein can be isolated from placental perfusate by any technique known in the art, and said placental perfusate can be collected by any technique known in the art. In certain embodiments, placental perfusate is collected from a pre-term mammalian placenta. In certain embodiments, placental perfusate is collected from a post-partum mammalian placenta. In some embodiments, placental perfusate is obtained from a post-partum mammalian placenta of a full-term birth. In other embodiments, placental perfusate is obtained from a post-partum mammalian placenta of a premature birth. The placental perfusate can be allogeneic to the infant (e.g., premature infant or full-term infant) to be treated, if administered alone, or can be allogeneic or autologous, or a combination of both, if administered with umbilical cord blood or blood additives. In a specific embodiment, the placental perfusate used in the methods described herein is autologous to the infant being treated.

Placental stem cells used in the methods described herein can be isolated and processed by any method known in the art. In certain embodiments, placental stem cells are obtained from a placenta of a full-term birth. In certain embodiments, placental stem cells are obtained from a placenta of a premature birth. The placental stem cells useful in the methods described herein can be allogeneic or autologous to the premature infant to be treated, or a combination of both. In a specific embodiment, the placental stem cells used in the methods described herein are autologous to the infant being treated. In another specific embodiment, the placental stem cells used in the methods described herein are placental stem cells contained within, or derived from, placental perfusate, as described in detail below.

In particular embodiments, cells obtained from placental perfusate (e.g., total nucleated cells from placental perfusate) are obtained from a placenta that has been exsanguinated and perfused to remove residual blood cells. In one embodiment, cells are obtained from placental perfusate (e.g., total nucleated cells from placental perfusate) by isolating total nucleated cells, or a subset thereof, from other components of perfusate such as, e.g., enucleated cells, cell debris, or serum. In certain embodiments, cells obtained from placental perfusate to be used in accordance with the methods described herein comprise cells with the following cell surface markers: CD34⁺CD45⁻, CD34⁺CD45⁺, CD105⁺CD200⁺, and/or CD34⁺CD45⁺CD41⁺CD61⁺.

In particular embodiments, placental stem cells are obtained from a placenta that has been exsanguinated and perfused to remove residual blood cells. The exsanguinated placenta can then be cultured for about 2 to about 24 hours, or more, under conditions appropriate to allow for the production of endogenous stem cells originating from the placenta. Once obtained from a cultured placenta, the placental stem cells may be characterized by a number of methods, including but not limited to, immunochemistry to identify particular cell surface markers. In certain embodiments, placental stem cells to be used in accordance with the methods described herein may be identified by the presence of the following cell surface markers: CD34⁻, OCT-4⁺, CD73⁺, CD105⁺, CD200⁺ and/or HLA-G⁺. In certain embodiments, the placental stem cells comprise CD34⁺ cells. In certain embodiments, the placental stem cells comprise CD34⁻ cells. In certain embodiments, the placental stem cells comprise OCT-4⁺ cells. In certain embodiments, the placental stem cells comprise cells that are CD73⁺, CD105⁺ and CD200⁺. In certain embodiments, the placental stem cells comprise cells that are CD200⁺ or OCT-4⁺. In certain embodiments, said placental stem cells comprise cells that are CD200⁺ and OCT-4⁺. In certain embodiments, the placental stem cells comprise cells that are CD73⁺ and CD105⁺ and that facilitate the formation of one or more embryoid-like bodies in a population of placental cells comprising said cells when said population is cultured under conditions that allow the formation of an embryoid-like body. In certain embodiments, the placental stem cells comprise cells that are CD73⁺, CD105⁺ and CD200⁺. In certain embodiments, the placental stem cells comprise cells that are OCT-4⁺ and that facilitates the formation of one or more embryoid-like bodies in a population of placental cells comprising the stem cell when said population is cultured under conditions that allow formation of embryoid-like bodies. In a specific embodiment, the placental stem cells are CD34⁻, CD10⁺, CD105⁺, CD200⁺ placental stem cells.

The step of administering to the infants (e.g., full-term and premature infants) cells obtained from placental perfusate (e.g., total nucleated cells from placental perfusate), placental stem cells and/or umbilical cord blood or cells obtained therefrom (e.g., total nucleated cells from umbilical cord blood), or combinations of umbilical cord blood or cells obtained therefrom (e.g., total nucleated cells from umbilical cord blood) and cells obtained from placental perfusate (e.g., total nucleated cells from placental perfusate) or placental stem cells, can be carried out according to the judgment of those of skill in the art, and can be performed, e.g., intravenously, subcutaneously, locally, systemically, intraperitoneally, parenterally, or intracerebroventricularly (ICV). Administration can be performed at various times after birth of the infant (e.g., premature infant or full-term infant), e.g., no more than about 2 weeks after birth. In various embodiments, administration is performed within one, two, five, ten, twelve, or twenty four hours, or one week, after birth of the infant (e.g., premature infant or full-term infant). Administration can be performed once or a plurality of times after birth of the infant (e.g., premature infant or full-term infant).

### 4. BRIEF DESCRIPTION OF THE DRAWINGS

FIG. 1 is a cross-sectional view of the cannulation of the vein and artery of a placenta to perfuse the placenta and then collect the perfusate.

FIGS. 2A-2E are schematics showing the collection, clamping, perfusion, collection and storage of a drained and perfused placenta.

FIG. 3 is a cross-sectional schematic of a perfused placenta in a device for use as a bioreactor.

FIG. 4 depicts survival rates of rats in which focal cerebral ischemia (FCI) was induced following treatment with placental stem cells (PSC) or vehicle control (DMEM).

FIG. 5 depicts stance width of rats in which focal cerebral ischemia was induced following treatment with placental stem cells (FCI+low dose; FCI+high dose) or vehicle control (FCI control). Also depicted is the stance width of sham control rats, which underwent surgery without induction of FCI.

### 5. DETAILED DESCRIPTION

### 5.1 Treatment of Infants

Provided herein are methods of treating disorders and conditions of infants (e.g., premature infants and full-term infants), including disorders and conditions of premature infants caused by or associated with premature birth.

In certain embodiments, the methods comprise the step of administering to the infant (e.g., premature infant or full-term infant) umbilical cord blood or cells obtained therefrom (e.g., total nucleated cells from umbilical cord blood) and, optionally, cells obtained from placental perfusate (e.g., total nucleated cells from placental perfusate) or placental stem cells. As used herein, in the context of umbilical cord blood, “cells obtained from umbilical cord blood” or “cells obtained therefrom” comprise total nucleated cells obtained from, e.g., isolated from, umbilical cord blood, a subset of nucleated cells obtained from umbilical cord blood, or cells cultured or proliferated from cells obtained directly from umbilical cord blood. In a particular embodiment, the cells obtained from umbilical cord blood for use in accordance with the methods described herein are total nucleated cells obtained from, e.g., isolated from, umbilical cord blood. As used herein, “cells obtained from placental perfusate” comprise total nucleated cells obtained from, e.g., isolated from, placental perfusate, a subset of nucleated cells obtained from placental perfusate, or cells cultured or proliferated from cells obtained directly from placental perfusate. In a particular embodiment, the cells obtained from placental perfusate for use in accordance with the methods described herein are total nucleated cells obtained from, e.g., isolated from, placental perfusate. In certain embodiments, in which the cord blood is administered alone or with blood additives, the cord blood is allogeneic to the infant (e.g., premature infant or full-term infant) to be treated. In certain embodiments, in which the cord blood is administered with cells obtained from placental perfusate (e.g., total nucleated cells from placental perfusate), placental stem cells and/or blood additives, the cord blood can be autologous or allogeneic to the recipient infant (e.g., premature infant or full-term infant). In a specific embodiment, the cord blood administered to the infant (e.g., premature infant or full-term infant) is autologous to the infant.

In certain embodiments, the umbilical cord blood or cells obtained therefrom (e.g., total nucleated cells from umbilical cord blood) administered in accordance with the methods described herein is from a single umbilical cord. In certain embodiments, the umbilical cord blood or cells obtained therefrom (e.g., total nucleated cells from umbilical cord blood) administered in accordance with the methods described herein is from more than one umbilical cord. In certain embodiments, the umbilical cord blood or cells obtained therefrom (e.g., total nucleated cells from umbilical cord blood) administered in accordance with the methods described herein is from two umbilical cords. In certain embodiments, the cells obtained from placental perfusate (e.g., total nucleated cells from placental perfusate) administered in accordance with the methods described herein are from a single placenta. In certain embodiments, the cells obtained from placental perfusate (e.g., total nucleated cells from placental perfusate) or a composition comprising cells obtained from placental perfusate administered in accordance with the methods described herein are from more than one placenta. In certain embodiments, the cells obtained from placental perfusate (e.g., total nucleated cells from placental perfusate) or a composition comprising cells obtained from placental perfusate administered in accordance with the methods described herein are from two placentas. In certain embodiments, the placental stem cells or composition comprising placental stem cells administered in accordance with the methods described herein are from a single placenta. In certain embodiments, the placental stem cells or composition comprising placental stem cells administered in accordance with the methods described herein are from more than one placenta. In certain embodiments, the placental stem cells or composition comprising placental stem cells administered in accordance with the methods described herein are from two placentas.

In certain embodiments, the methods comprise the step of administering to the infant (e.g., premature infant or full-term infant) a composition comprising cells obtained from placental perfusate (e.g., total nucleated cells from placental perfusate). In a specific embodiment, the placental perfusate is autologous, i.e., the placental perfusate is derived from the placenta of the infant being treated.

In certain embodiments, the methods comprise the step of administering to the infant (e.g., premature infant or full-term infant) a composition comprising placental stem cells. In a specific embodiment, the placental stem cells are autologous placental stem cells, i.e., the placental stem cells are derived from the placenta of the infant being treated. In another specific embodiment, the placental stem cells administered are contained within, or are derived from, placental perfusate.

In a specific embodiment, provided herein is a method of treating a disorder or condition of a infant (e.g., premature infant or full-term infant), comprising administering to the infant a composition comprising umbilical cord blood or cells obtained therefrom (e.g., total nucleated cells from umbilical cord blood) and cells obtained from placental perfusate (e.g., total nucleated cells from placental perfusate). In a specific embodiment, either the cord blood or the placental perfusate are autologous to the infant. In another specific embodiment, both the cord blood and the placental perfusate (e.g., total nucleated cells from placental perfusate) are autologous to the infant.

In a specific embodiment, provided herein is a method of treating a disorder or condition of a infant (e.g., premature infant or full-term infant), comprising administering to the infant a composition comprising umbilical cord blood or cells obtained therefrom (e.g., total nucleated cells from umbilical cord blood) and placental stem cells. In a specific embodiment, either the cord blood or the placental stem cells are autologous to the infant. In another specific embodiment, both the cord blood and the placental stem cells are autologous to the infant. In certain embodiments, the placental stem cells administered are placental stem cells contained within, or derived from, placental perfusate.

An infant is considered an extremely low birth weight (ELBW) infant if the infant is born weighing less than 1 kg (approximately 2.3 lbs). In certain embodiments, the infant (e.g., premature infant) treated in accordance with the methods described herein weighs about 800 grams or more at birth. In certain embodiments, the infant (e.g., premature infant) treated in accordance with the methods described herein weighs at about 500 grams to about 800 grams at birth. In certain embodiments, the infant (e.g., premature infant) treated in accordance with the methods described herein weighs less than about 500 grams at birth.

As used herein, the terms “treat,” “treating” and “treatment” refer to the cure, remediation, or the reduction or amelioration of the progression, severity, and/or duration, of a disorder or condition, for example, one caused by or related to premature birth, or any parameter or symptom of such a disorder or condition.

Treatment of an infant (e.g., premature infant or full-term infant) with umbilical cord blood or cells obtained therefrom (e.g., total nucleated cells from umbilical cord blood), and optionally cells obtained from placental perfusate (e.g., total nucleated cells from placental perfusate) or placental stem cells, or with cells obtained from placental perfusate (e.g., total nucleated cells from placental perfusate) or placental stem cells alone, may be considered efficacious if the infant (e.g., premature infant or full-term infant) survives, or if the disorder or condition to be treated (e.g., disease or disorder caused by or associated with premature birth) is measurably improved in any way as a result of the treatment. Such improvement may be shown by, e.g., one or more measurable indicators including, for example, detectable changes in a physiological condition or set of physiological conditions associated with a particular disease, disorder or condition (including, but not limited to, blood pressure, heart rate, respiratory rate, counts of various blood cell types, levels in the blood of certain proteins, carbohydrates, lipids or cytokines or modulation of expression of genetic markers associated with the disease, disorder or condition).

Treatment of an infant (e.g., premature infant or full-term infant) with umbilical cord blood or cells obtained therefrom (e.g., total nucleated cells from umbilical cord blood), and optionally cells obtained from placental perfusate (e.g., total nucleated cells from placental perfusate) or placental stem cells, or with cells obtained from placental perfusate (e.g., total nucleated cells from placental perfusate) or placental stem cells alone, is considered effective if any one of such indicators appears to respond to such treatment by changing to a value that is within, or closer to, a normal value for, e.g. healthy full-term infants, than such indicator(s) would be expected to lie in the absence of administration of umbilical cord blood or cells obtained therefrom (e.g., total nucleated cells from umbilical cord blood) and/or cells obtained from placental perfusate (e.g., total nucleated cells from placental perfusate) or placental stem cells. The normal value may be, a normal value or range of normal values that is known in the art for an indicator. For example, one or more metabolic or biochemical indicator displayed by an infant (e.g., premature infant or full-term infant) can be compared to the normal range for the indicator(s), wherein treatment is considered effective if the treatment results in the one or more metabolic or biochemical indicator more closely approaching, or falling within, a reference range for normal, full-term infants. Such indicators include, but are not limited to, levels of, or values for, 17 hydroxyprogesterone, 25-hydroxyvitamin D (25(OH)D), acetoacetate, acidity (pH), albumin, ammonia, amylase, ascorbic acid, bicarbonate, bilirubin, blood volume, calcium, carbon, dioxide partial pressure, carbon monoxide, CD4 cell count, ceruloplasmin, chloride, copper, creatine kinase (CK or CPK), creatine kinase isoenzymes, creatinine, erythrocyte sedimentation rate (ESR or Sed-Rate), globulin, glucose, hematocrit, hemoglobin, iron, iron-binding capacity, lactate (lactic acid) (arterial), lactic dehydrogenase, lipase, magnesium, mean corpuscular hemoglobin (MCH), mean corpuscular hemoglobin concentration (MCHC), mean corpuscular volume (MCV), osmolality, oxygen partial pressure, oxygen saturation (arterial), phosphatase, phosphorus, platelet count, potassium, protein (total), prothrombin (PTT), pyruvic acid, red blood cell count (RBC), sodium, thyroid-stimulating hormone (TSH), transaminase (alanine or aspartate), urea nitrogen (BUN) and BUN/creatinine ratio, uric acid, vitamin A, WBC (leukocyte count and white blood cell count), zinc, and the like.

The effectiveness of administration of cord blood or cells obtained therefrom (e.g., total nucleated cells from umbilical cord blood), or cord blood or cells obtained therefrom (e.g., total nucleated cells from umbilical cord blood) and cells obtained from placental perfusate (e.g., total nucleated cells from placental perfusate) or placental stem cells, or a composition comprising cells obtained from placental perfusate (e.g., total nucleated cells from placental perfusate) or placental stem cells, can be assessed by behavioral tests. For example, improvement in neurodevelopment of an infant (e.g., premature infant or full-term infant) can be assessed, e.g., within the first 2-3 years of birth by one or more such tests as the Neonatal Neurobehavioral Examination, Alberta Infant Motor Scale, or Bayley Scale of infant Development (3d Edition), by comparing a score on such a test by the premature infant to a score, or range of scores, for normal and premature infants of different gestational ages, and determining that improvement takes place if the score is higher than the score is expected to be at the premature infant's gestational age. In a specific embodiment, the infant (e.g., premature infant or full-term infant) receiving cord blood or cells obtained therefrom (e.g., total nucleated cells from umbilical cord blood), cells obtained from placental perfusate (e.g., total nucleated cells from placental perfusate) or placental stem cells, or a combination of cord blood or cells obtained therefrom (e.g., total nucleated cells from umbilical cord blood) and cells obtained from placental perfusate (e.g., total nucleated cells from placental perfusate) or placental stem cells, is deemed to have improved if the score is higher than a score, or an average of scores, of infants of the same gestational age treated with only conventional treatments.

In one embodiment, an infant (e.g., premature infant or full-term infant) is assessed shortly after birth (e.g., within the first week) by the Neonatal Neurobehavioral Examination (NNE) and given a score representing development of behavioral traits, primitive reflexes, and tone and motor patterns, wherein the maximum score is 81. The infant is re-assessed one or more times within two years of birth, e.g., at about 18 to about 22 months. A significant improvement in the NNE score during this time indicates efficacy. In various embodiments, administration of umbilical cord blood or cells obtained therefrom (e.g., total nucleated cells from umbilical cord blood) and optionally cells obtained from placental perfusate (e.g., total nucleated cells from placental perfusate) or placental stem cells, or a composition comprising cells obtained from placental perfusate or placental stem cells, is effective if the score improves from average scores of, e.g., 37-42 week gestational age preterm infants (66.5) if the premature infant was born at 37-42 weeks gestational age; 34-36 week preterm infants (60.7) if the premature infant was born at 34-36 weeks gestational age, or infants born at 34 weeks or less gestational age (51.1) if the premature infant was born at 34 weeks or less gestational age, compared with premature infants not treated with cord blood or cells obtained therefrom (e.g., total nucleated cells from umbilical cord blood) or a combination of cord blood or cells obtained therefrom (e.g., total nucleated cells from umbilical cord blood) and cells obtained from placental perfusate (e.g., total nucleated cells from placental perfusate) or placental stem cells. See Morgan, “Neonatal Neurobehavioral Examination. A New Instrument For Quantitative Analysis of Neonatal Neurological Status,” Phys. Ther. 68(9):1352-1358 (1988).

### 5.1.1 Disorders or Conditions

The disorder or condition to be treated herein can be any disorder or condition, that is known in the art, including any disease or disorder caused by or associated with premature birth. In certain embodiments, the disorder or condition is Respiratory Distress Syndrome (RDS) or Acute Respiratory Distress Syndrome (ARDS). In certain embodiments, the disorder or condition is anemia. In certain embodiments, the disorder or condition is intraventricular hemorrhage, necrotizing enterocolitis, retinopathy of prematurity, chronic lung disease (bronchopulmonary dysplasia), an infection, patent ductus arteriosus, apnea, low blood pressure, or hyperbilirubinemia. In certain embodiments, the disorder or condition is caused by incomplete development of an organ, including but not limited to lung, eye, immune system, brain, heart, liver or kidney. In certain embodiments, the disorder or condition is caused by ischemia, e.g., ischemia in the brain.

(Acute) Respiratory Distress Syndrome (ARDS/RDS) or Infant Respiratory Distress Syndrome (IRDS) (formerly called hyaline membrane disease) is a breathing disorder in which the air sacs (alveoli) in an infant's lungs do not stay open because of high surface tension resulting from insufficient production of surfactant. Respiratory distress syndrome affects 10% of all premature infants and only rarely affects those born at full-term. The disease is caused by a lack of lung surfactant, a chemical that normally appears in mature lungs. Surfactant keeps the air sacs from collapsing and allows them to inflate with air more easily. In respiratory distress syndrome, the air sacs collapse and prevent the child from breathing properly. Symptoms usually appear shortly after birth and become progressively more severe. Infants with RDS/ARDS usually need support with oxygen and a respirator or are treated with a surfactant drug.

Anemia is a disorder characterized by an insufficient number of erythrocytes or hemoglobins in the blood to carry adequate oxygen to the body. Infants, e.g., premature infants, may develop anemia for a number of reasons, including blood loss during delivery, lack of iron content and shorter half-life of red blood cells as compared to adults. Current treatment options include blood transfusion (from blood bank or directed donor blood obtained from family members), iron supplementation and preventing of blood loss. Although not intending to be bound by any particular theory, it is believed that umbilical cord blood can be a good source of blood for transfusion. Autologous infusion of umbilical cord blood has several advantages over blood from a blood bank or related donors: 1) umbilical cord blood is a good source of erythrocytes with moderate capacity to carry oxygen; 2) umbilical cord blood is readily available and requires minimal testing; and 3) the umbilical cord blood is a rich source of stem and progenitor cells capable of supporting further development of immature organs or organs damaged due to premature delivery.

Apnea is a disorder in which an infant, e.g., premature infant, temporarily stops breathing and is usually defined as cessation of breathing for 15 to 20 seconds. Apnea may occur in infants born before 34 weeks of pregnancy, increasing in frequency among the most prematurely born infants. It is thought to be caused by immaturity of the part of the brain that controls breathing. Infants with apnea are treated with drugs (e.g., aminophylline, caffeine, or doxapram), and/or are supported with continuous positive airway pressure or a ventilator.

Chronic lung disease (bronchopulmonary dysplasia) is a condition that develops in infants, e.g., premature infants, on mechanical ventilation and/or high oxygen levels for extended periods. Chronic lung disease is treated with oxygen, drugs and gradual weaning infants from the ventilator.

Hyperbilirubinemia, one of the most common problems encountered in newborns, is an abnormally high level of bilirubin in the blood. It is defined as a total serum bilirubin level greater than 5 mg/dL. Bilirubin above this level is neurotoxic/cytotoxic. It results from the deposition of unconjugated bilirubin pigment in the skin and mucus membranes. Mild hyperbilrubinemia does not require treatment. Higher bilirubin levels can be treated by phototherapy, in which the infant is placed under bilirubin lights.

Premature infants are at higher risk of developing infections than full-term babies, since their immune systems are immature. Serious infections seen in premature babies include sepsis, pneumonia and meningitis (infection of the membranes surrounding the brain). Currently, infections are treated with antibiotics or antiviral drugs.

Intraventricular hemorrhage is bleeding in the brain. It occurs when small blood vessels lying alongside the ventricles rupture. Intraventricular hemorrhage most frequently affects premature newborns Severe bleeding can cause brain damage, and can result in, e.g., learning disabilities and/or behavior problems.

Necrotizing enterocolitis is a condition in which the inner surface of the intestine becomes injured and inflamed; if severe, a portion of the intestine may die, leading to intestinal perforation and peritonitis. Necrotizing enterocolitis occurs mainly in premature infants. The cause for this disorder is unknown, but it is thought that a decreased blood flow to the bowel keeps the bowel from producing the normal protective mucus. Bacteria in the intestine may also be a cause. Necrotizing enterocolitis can lead to feeding problems, swelling in infants' belly and other complications. Necrotizing enterocolitis is treated with drugs and sometimes surgery.

Patent ductus arteriosus (PDA) is a condition where the ductus arteriosus, a blood vessel that allows blood to bypass the infant's lungs before birth, fails to close after birth. Patent ductus arteriosus is currently treated with drugs and surgery if necessary.

Retinopathy of prematurity is a disorder in which blood vessels in the back of the eye (retina) develop abnormally in premature infants; these blood vessels may bleed, and in the most severe cases, the retina may detach, leading to visual loss. It occurs mainly in infants born before the 32d week of pregnancy. The main risk factor for developing retinopathy of prematurity is extreme prematurity; high oxygen levels in the blood from the treatment of breathing problems may increase the risk. A bilateral retinopathy typically occurring in premature infants treated with high concentrations of oxygen, characterized by vascular dilatation, proliferation, and tortuosity, edema, and retinal detachment, with ultimate conversion of the retina into a fibrous mass that can be seen as a dense retrolental membrane. Typically, growth of the eye is arrested and may result in microphthalmia, and blindness may occur. Mild retinopathy of prematurity often heals spontaneously; infants with severe retinopathy are currently treated with a laser or cryotherapy, in which the peripheral portions of the retina are frozen.

In certain embodiments, the disease or disorder treated in accordance with the methods described herein is a neurological disorder. Neurological disorders that can be treated using the methods described herein include, without limitation, intraventricular hemorrhage, hypoxic ischemic encephalopathy, retinopathy of prematurity, cerebral palsy, and white matter brain abnormalities. In a specific embodiment, the methods described herein are used to treat an infant with a neurological disorder, wherein said neurological disorder is caused by ischemia.

In certain embodiments, the disease or disorder treated in accordance with the methods described herein is a gastrointestinal disorder. Gastrointestinal disorders that can be treated using the methods described herein include, without limitation, necrotizing enterocolitis and gastroesophageal reflux disease (GERD).

In certain embodiments, the disease or disorder treated in accordance with the methods described herein is a respiratory disorder. Respiratory disorders that can be treated using the methods described herein include, without limitation, respiratory distress syndrome, apnea (e.g., apnea of prematurity), and bronchopulmonary dysplasia.

Also encompassed herein is the treatment of disorders and conditions caused by incomplete development of certain organs, including but not limited to lung, eye, immune system, brain, heart, liver and kidney.

### 5.1.2 Administration of Umbilical Cord Blood or Cells Obtained from Umbilical Cord Blood and/or Cells Obtained from Placental Perfusate or Placental Stem Cells

Umbilical cord blood or cells obtained therefrom (e.g., total nucleated cells from umbilical cord blood), and optionally cells obtained from placental perfusate (e.g., total nucleated cells from placental perfusate) or placental stem cells, or a composition comprising cells obtained from placental perfusate or placental stem cells, may be administered to an infant, e.g., a full-term or premature infant, in any pharmaceutically or medically acceptable manner, including by injection or transfusion. In a specific embodiment, umbilical cord blood or cells obtained therefrom (e.g., total nucleated cells from umbilical cord blood) and/or cells obtained from placental perfusate or placental stem cells are administered to a premature infant having or exhibiting any disorder or condition associated with, or caused by, prematurity. In certain embodiments, umbilical cord blood or cells obtained therefrom (e.g., total nucleated cells from umbilical cord blood) and/or cells obtained from placental perfusate (e.g., total nucleated cells from placental perfusate), or placental stem cells are administered to an infant (e.g., a premature infant or full-term infant) parenterally. The term “parenteral” as used herein includes subcutaneous injections, intravenous, intramuscular, intra-arterial injection, or infusion techniques. In certain embodiments, umbilical cord blood or cells obtained therefrom (e.g., total nucleated cells from umbilical cord blood), and optionally cells obtained from placental perfusate (e.g., total nucleated cells from placental perfusate) or placental stem cells, or a composition comprising cells obtained from placental perfusate or placental stem cells, are administered to a premature infant intravenously. In certain embodiments umbilical cord blood or cells obtained therefrom (e.g., total nucleated cells from umbilical cord blood) and/or cells obtained from placental perfusate (e.g., total nucleated cells from placental perfusate) or placental stem cells are administered to an infant (e.g., a premature infant or full-term infant) intraventricularly. In certain embodiments, umbilical cord blood or cells obtained therefrom (e.g., total nucleated cells from umbilical cord blood) and/or cells obtained from placental perfusate (e.g., total nucleated cells from placental perfusate) or placental stem cells are administered to an infant (e.g., a premature infant or full-term infant) locally (e.g., at the site of injury). In certain embodiments, umbilical cord blood or cells obtained therefrom (e.g., total nucleated cells from umbilical cord blood) and/or cells obtained from placental perfusate (e.g., total nucleated cells from placental perfusate) or placental stem cells are administered to an infant (e.g., a premature infant or full-term infant) systemically.

Umbilical cord blood or cells obtained therefrom (e.g., total nucleated cells from umbilical cord blood), cells obtained from placental perfusate (e.g., total nucleated cells from placental perfusate), or placental stem cells may be contained in any pharmaceutically-acceptable carrier. The umbilical cord blood or cells obtained therefrom (e.g., total nucleated cells from umbilical cord blood), cells obtained from placental perfusate (e.g., total nucleated cells from placental perfusate), or placental stem cells may be carried, stored, or transported in any pharmaceutically or medically acceptable container, for example, a blood bag, transfer bag, plastic tube, syringe, vial, or the like.

The step of administering umbilical cord blood or cells obtained therefrom (e.g., total nucleated cells from umbilical cord blood), and optionally cells obtained from placental perfusate (e.g., total nucleated cells from placental perfusate) or placental stem cells, or a composition comprising cells obtained from placental perfusate or placental stem cells, to the infant (e.g., premature infant or full-term infant) can be carried out in any medically-acceptable manner. Administration can be performed at various times after birth of the infant (e.g., premature infant or full-term infant). In various embodiments, for example, administration is performed within 1, 2, 3, 4, 5, 6, 7, 8, 9, 10, 11, 12, 15, 18, 21, or 24 hours afterbirth, or within 2, 3, 4, 5, or 6 days after birth, or within 1 or 2 weeks after birth, or within 1, 2, 3, 4, 5, 6, 7, 8, 9, 10, 11, or 12 months after birth.

Administration of cord blood or cells obtained therefrom (e.g., total nucleated cells from umbilical cord blood), and optionally of cells obtained from placental perfusate (e.g., total nucleated cells from placental perfusate) or placental stem cells, or a composition comprising cells obtained from placental perfusate or placental stem cells, can be performed once or a plurality of times after birth of the infant (e.g., premature infant or full-term infant). In certain embodiments, administration is performed once after birth of an infant (e.g., premature infant or full-term infant). In certain embodiments, administration is performed a plurality of times after birth of an infant (e.g., premature infant or full-term infant).

In certain embodiments, the umbilical cord blood or cells obtained therefrom (e.g., total nucleated cells from umbilical cord blood) administered in accordance with the methods described herein is from a single umbilical cord. In certain embodiments, the umbilical cord blood or cells obtained therefrom (e.g., total nucleated cells from umbilical cord blood) administered in accordance with the methods described herein is from more than one umbilical cord. In certain embodiments, the umbilical cord blood or cells obtained therefrom (e.g., total nucleated cells from umbilical cord blood) administered in accordance with the methods described herein is from two umbilical cords. In certain embodiments, the cells obtained from placental perfusate (e.g., total nucleated cells from placental perfusate) administered in accordance with the methods described herein are from a single placenta. In certain embodiments, the cells obtained from placental perfusate (e.g., total nucleated cells from placental perfusate) or a composition comprising cells obtained from placental perfusate administered in accordance with the methods described herein are from more than one placenta. In certain embodiments, the cells obtained from placental perfusate (e.g., total nucleated cells from placental perfusate) or a composition comprising cells obtained from placental perfusate administered in accordance with the methods described herein are from two placentas. In certain embodiments, the placental stem cells or composition comprising placental stem cells administered in accordance with the methods described herein are from a single placenta. In certain embodiments, the placental stem cells or composition comprising placental stem cells administered in accordance with the methods described herein are from more than one placenta. In certain embodiments, the placental stem cells or composition comprising placental stem cells administered in accordance with the methods described herein are from two placentas.

In certain embodiments, the amount of cord blood or cells obtained therefrom (e.g., total nucleated cells from umbilical cord blood) administered to an infant in accordance with the methods described herein can be determined based on the number of cells present in the cord blood. The amount or number of umbilical cord blood or cells obtained therefrom (e.g., total nucleated cells from umbilical cord blood) and/or cells obtained from placental perfusate (e.g., total nucleated cells from placental perfusate) or placental stem cells administered to the infant (e.g., premature infant or full-term infant) depends on the source of umbilical cord blood or cells obtained therefrom (e.g., total nucleated cells from umbilical cord blood) and/or cells obtained from placental perfusate (e.g., total nucleated cells from placental perfusate) or placental stem cells used, the severity or nature of disorders or conditions to be treated, as well as age, body weight and physical condition of the infant (e.g., premature infant or full-term infant), etc. In certain embodiments, about 0.01 to about 0.1, about 0.1 to about 1, about 1 to about 10, about 10 to about 10², about 10² to about 10³, about 10³ to about 10⁴, about 10⁴ to about 10⁵, about 10⁵ to about 10⁶, about 10⁶ to about 10⁷, about 10⁷ to about 10⁸, or about 10⁸ to about 10⁹ umbilical cord blood cells (e.g., total nucleated cells from umbilical cord blood), cells obtained from placental perfusate (e.g., total nucleated cells from placental perfusate), placental stem cells, or umbilical cord blood cells and cells obtained from placental perfusate (e.g., total nucleated cells from placental perfusate) or placental stem cells per kilogram body weight of an infant (e.g., premature infant or full-term infant) are administered. In a specific embodiment, said umbilical cord blood cells are CD34⁺ cells. In another specific embodiment, said cells obtained from placental perfusate (e.g., total nucleated cells from placental perfusate) are CD34⁺ cells. In another specific embodiment, said placental stem cells are CD34⁺ cells. In another specific embodiment, said umbilical cord cells are autologous to the infant. In another specific embodiment, said placental perfusate is autologous to the infant. In another specific embodiment, said placental stem cells are autologous to the infant. In certain embodiments, at least about 10⁵ to about 10⁷ CD34⁺ cells per kilogram body weight are administered. Such CD34⁺ cells can be from cord blood alone, or can be from cord blood and placenta. In various embodiments, at least about 0.1, 1, 10, 10², 10³, 10⁴, 10⁵, 10⁶, 10⁷, 10⁸, or 10⁹ umbilical cord blood cells (e.g., total nucleated cells from umbilical cord blood), cells obtained from placental perfusate (e.g., total nucleated cells from placental perfusate), or placental stem cells, or umbilical cord blood cells and cells obtained from placental perfusate (e.g., total nucleated cells from placental perfusate) or placental stem cells per kilogram body weight of an infant (e.g., premature infant or full-term infant) are administered. In specific embodiments, at least about 0.5×10⁶, 1.0×10⁶, 1.5×10⁶, 2.0×10⁶, 2.5×10⁶, 3.0×10⁶, 5.0×10⁶, 1.0×10⁷, 1.5×10⁷, 2.0×10⁷, 2.5×10⁷, 3.0×10⁷, 3.5×10⁷, 4.0×10⁷, 4.5×10⁷, 5.0×10⁷, 5.5×10⁷, or 6.0×10⁷ umbilical cord blood cells (e.g., total nucleated cells from umbilical cord blood), cells obtained from placental perfusate (e.g., total nucleated cells from placental perfusate), placental stem cells, or umbilical cord blood cells and cells obtained from placental perfusate (e.g., total nucleated cells from placental perfusate) or placental stem cells per kilogram body weight of an infant (e.g., premature infant or full-term infant) are administered. In a more specific embodiment, at least about 0.5×10⁶, 1.0×10⁶, 1.5×10⁶, 2.0×10⁶, 2.5×10⁶, or 3.0×10⁶ cells obtained from placental perfusate (e.g., total nucleated cells from placental perfusate) per kilogram body weight of an infant (e.g., premature infant or full-term infant) are administered. In a more specific embodiment, at least about 1.5×10⁷, 2.0×10⁷, 2.5×10⁷, 3.0×10⁷, 3.5×10⁷, 4.0×10⁷, 4.5×10⁷, 5.0×10⁷, 5.5×10⁷, or 6.0×10⁷ umbilical cord blood cells (e.g., total nucleated cells from umbilical cord blood) per kilogram body weight of an infant (e.g., premature infant or full-term infant) are administered. In various embodiments, at most about 10⁴, 10⁵, 10⁶, 10⁷, 10⁸, or 10⁹ umbilical cord blood cells, cells obtained from placental perfusate (e.g., total nucleated cells from placental perfusate), or placental stem cells, or umbilical cord blood cells and cells obtained from placental perfusate (e.g., total nucleated cells from placental perfusate) or placental stem cells per kilogram body weight of an infant (e.g., premature infant or full-term infant) are administered. In specific embodiments, at most about 0.5×10⁶, 1.0×10⁶, 1.5×10⁶, 2.0×10⁶, 2.5×10⁶, 3.0×10⁶, 5.0×10⁶, 1.0×10⁷, 1.5×10⁷, 2.0×10⁷, 2.5×10⁷, 3.0×10⁷, 3.5×10⁷, 4.0×10⁷, 4.5×10⁷, 5.0×10⁷, 5.5×10⁷, or 6.0×10⁷ umbilical cord blood cells (e.g., total nucleated cells from umbilical cord blood), cells obtained from placental perfusate (e.g., total nucleated cells from placental perfusate), placental stem cells, or umbilical cord blood cells and cells obtained from placental perfusate (e.g., total nucleated cells from placental perfusate) or placental stem cells per kilogram body weight of an infant (e.g., premature infant or full-term infant) are administered. In a more specific embodiment, at most about 0.5×10⁶, 1.0×10⁶, 1.5×10⁶, 2.0×10⁶, 2.5×10⁶, or 3.0×10⁶ cells obtained from placental perfusate (e.g., total nucleated cells from placental perfusate) per kilogram body weight of an infant (e.g., premature infant or full-term infant) are administered. In a more specific embodiment, at most about 1.5×10⁷, 2.0×10⁷, 2.5×10⁷, 3.0×10⁷, 3.5×10⁷, 4.0×10⁷, 4.5×10⁷, 5.0×10⁷, 5.5×10⁷, or 6.0×10⁷ umbilical cord blood cells (e.g., total nucleated cells from umbilical cord blood) per kilogram body weight of an infant (e.g., premature infant or full-term infant) are administered. In specific embodiments of the above embodiments, the cord blood cells (e.g., total nucleated cells from umbilical cord blood), cells obtained from placental perfusate (e.g., total nucleated cells from placental perfusate) and/or placental stem cells are CD34⁺ cells. In another specific embodiment, the cord blood cells comprise CD34⁺ cells and the cells obtained from placental perfusate (e.g., total nucleated cells from placental perfusate) are CD34⁺CD45⁻, CD34⁺CD45⁺, CD105⁺CD200⁺, or CD34⁺CD45⁺CD41⁺CD61⁺. In another specific embodiment, the cord blood cells comprise CD34⁺ cells and the placental stem cells comprise CD34⁻, CD10⁺, CD105⁺, CD200⁺ placental stem cells. In another specific embodiment, the placental stem cells administered are placental stem cells contained within, or derived from, placental perfusate.

The umbilical cord blood or cells obtained therefrom (e.g., total nucleated cells from umbilical cord blood), and optionally cells obtained from placental perfusate (e.g., total nucleated cells from placental perfusate) or placental stem cells, or a composition comprising cells obtained from placental perfusate or placental stem cells, can be delivered in a volume appropriate for the size of the infant (e.g., premature or full-term infant). Typical blood volume of premature infants is about 85-100 mL/kg body weight. Thus, the blood volume for premature infants ranges from approximately 40 mL to approximately 300 mL. In various embodiments, therefore, umbilical cord blood or cells obtained therefrom (e.g., total nucleated cells from umbilical cord blood), and optionally cells obtained from placental perfusate (e.g., total nucleated cells from placental perfusate) or placental stem cells, or a composition comprising cells obtained from placental perfusate or placental stem cells, is administered in a total volume of about 0.5 mL, 1.0 mL, 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, or about 20 mL, or more. The administration of such volumes can be a single administration or in multiple administrations. Where the infant is particularly low birth weight (e.g., less than 1 kg), a desired volume of umbilical cord blood, or number of umbilical cord blood cells, and optionally cells obtained from placental perfusate (e.g., total nucleated cells from placental perfusate) or placental stem cells, can be provided to the infant in a plurality of administrations. The time over which such volumes of cord blood or number of cord blood cells or combinations of cord blood or cord blood cells and cells obtained from placental perfusate (e.g., total nucleated cells from placental perfusate) or placental stem cells can be administered can vary from, e.g., 0.5 hours, 1 hour, 1.5 hours, 2 hours, 2.5 hours, 3 hours, 3.5 hours, 4 hours, or more.

Generally, small transfusions under 20 mL do not require a pump and may be pushed in via a syringe by, e.g., an intermittent small bolus, taking into consideration the volume-tolerance of the infant. Larger-volume transfusions can administered by an infusion device, within a period of two to four hours. If the transfusion interval is to exceed four hours, the blood component should be subdivided, and its second portion stored, e.g. in a blood bank, until needed.

### 5.1.3 Blood Additives

In certain embodiments, the cord blood (or cells obtained therefrom, e.g., total nucleated cells from umbilical cord blood) administered to the infant (e.g., premature or full-term infant) comprises one or more additives. For example, such additives can include, e.g., erythropoietin, an iron supplement, a vitamin, or allogeneic red blood cells.

In a specific embodiment, the blood additive is erythropoietin. Erythropoietin can be administered in any medically-acceptable form. The erythropoietin can be native erythropoietin purified from a biological source, or an engineered erythropoietin such as EPOGEN®, ARANESP® or PROCRIT®. The erythropoietin can have the sequence of native human erythropoietin, or can be an erythropoietin engineered to increase serum half-life. Typical dosages for erythropoietin range from 500-1500 U/kg/week.

In another specific embodiment, the blood additive is iron or an iron supplement. The iron or iron supplement can be in any metabolically available and medically acceptable form, e.g., elemental iron. Typical dosages for preterm infants are from about 4.0 mg/kg/day to about 4.5 mg/kg/day, where the preterm infant weighs about 1500 grams or less, and about 2 mg/kg/day where the infant weighs about 1500 grams to about 2500 grams.

In another specific embodiment, the blood additive is, or comprises, one or more vitamins. In certain embodiments, the one or more vitamins are those vitamins necessary for blood development, including, but not limited to, riboflavin (vitamin B2), pyridoxine (vitamin B6), folic acid, vitamin B12, and/or vitamin E. In one embodiment, about 25 IU of vitamin E is administered to the premature infant. Such vitamins can be administered to the premature infant at approximately the same dosage, per kilogram, as established for adults. In various embodiments, a single administration of cord blood and, optionally, cells obtained from placental perfusate (e.g., total nucleated cells from placental perfusate) or placental stem cells, includes about 150 μg riboflavin; about 100 μg pyridoxine; about 25 μg folic acid; about 0.4 μg vitamin B12; and/or about 3.6 IU vitamin E. The blood additive can also be one or more other vitamins, e.g., vitamin A (e.g., about 460 IU per administration); vitamin D (e.g., about 70 IU per administration); vitamin K (e.g., about 11 μg per administration); thiamin (vitamin B1, e.g., about 220 μg per administration); niacin (e.g., about 1950 μg per administration); pantothenic acid (e.g., about 800 μg per administration); biotin (e.g., about 9 μg per administration); vitamin C (ascorbic acid, e.g., about 15 mg per administration); inositol (e.g., about 6 mg per administration); and/or linoleic acid (e.g., about 750 mg per administration). The blood additive can be, or can comprise, any combination of the foregoing vitamins.

In another specific embodiment, the blood additive is allogeneic red blood cells from a source other than cord blood, e.g., red blood cells from peripheral blood. In one embodiment, where the donor is a first- or second-degree relative, the red blood cells are irradiated to a degree sufficient to reduce or prevent graft versus host disease. For example, in one embodiment, red blood cells are irradiated with at least 2,500 cGy prior to administration. Such irradiated red blood cells can be administered within 24 hours of irradiation. In one embodiment, the red blood cells administered to the infant (e.g., premature or full-term infant) with the cord blood are from an unrelated donor. In certain embodiments, such red blood cells are from a single unrelated donor. In one embodiment, the red blood cells are collected from the unrelated donor using a multipack blood collection system, which enables multiple administrations to the premature infant from the same donor, reducing immunological complications. In another more specific embodiment, wherein said additive is allogeneic red blood cells, said cells have been leukodepleted, e.g., using a leukodepletion filter. See, e.g., U.S. Pat. No. 5,728,306. In all embodiments in which red blood cells are used as a blood additive, the red blood cells can have been stored for no more than five days prior to administration. In one embodiment, the red blood cells comprise adenine-saline (AS-3) anticoagulant.

In another embodiment, the red blood cells are from units of donated blood that have been tested for the absence of at least the following pathogens, or absence of antibodies to the following pathogens: human immunodeficiency virus (HIV)-I, HIV-II, hepatitis C virus, human T-lymphotrophic virus (HTLV)-I and HTLV-II, hepatitis B virus (HBV, as evidenced, e.g., by absence of hepatitis B virus surface antigen (HBsAg)), cytomegalovirus, and syphilis.

### 5.1.4 Combination Therapy

In certain embodiments of the methods provided herein, umbilical cord blood or cells obtained therefrom (e.g., total nucleated cells from umbilical cord blood), and optionally cells obtained from placental perfusate (e.g., total nucleated cells from placental perfusate) or placental stem cells, or a composition comprising cells obtained from placental perfusate or placental stem cells, can be used as a first therapy in combination with one or more second therapies in the treatment of a disorder or condition of an infant (e.g., premature or full-term infant). Such second therapies include, but are not limited to, surgery, hormone therapy, immunotherapy, phototherapy or treatment with certain drugs.

The use of the term “combination therapy” does not limit the order in which treatments are administered to an infant (e.g., premature or full-term infant) in the methods provided. For example, the agents of the combination therapy can be administered concurrently, sequentially in any order or cyclically to a premature infant. In some embodiments, two components of a combination therapy are administered concurrently to an infant (e.g., premature or full-term infant).

Components of combination therapy can be administered to an infant (e.g., premature or full-term infant) in the same pharmaceutical composition. Alternatively, components of combination therapies can be administered to an infant (e.g., premature or full-term infant) in separate pharmaceutical compositions, and these separate compositions may be administered by the same or by different routes of administration, including, for example, oral, parenteral (e.g., ocular, nasal, dermal, muscular or peritoneal route(s), and the like), or topical, etc.

Particular second therapies are carried out according to the therapies' respective standard or art-recognized doses and dosing schedules.

In certain embodiments, a second therapeutic agent, and/or optional third therapeutic agent, is selected for its additive effects with umbilical cord blood or cells obtained therefrom (e.g., total nucleated cells from umbilical cord blood) and/or cells obtained from placental perfusate (e.g., total nucleated cells from placental perfusate) or placental stem cells in the treatment of a disorder or condition of an infant (e.g., premature or full-term infant).

In certain embodiments, a second therapeutic agent, and/or optional third therapeutic agent, is selected for its synergistic effects with umbilical cord blood or cells obtained therefrom (e.g., total nucleated cells from umbilical cord blood) and/or cells obtained from placental perfusate (e.g., total nucleated cells from placental perfusate) or placental stem cells in the treatment of a disorder or condition of an infant (e.g., premature or full-term infant).

Exemplary therapies that can be used in combination with administration of umbilical cord blood or cells obtained therefrom (e.g., total nucleated cells from umbilical cord blood), and optionally cells obtained from placental perfusate (e.g., total nucleated cells from placental perfusate) or placental stem cells, or with a composition comprising cells obtained from placental perfusate or placental stem cells, include control of environmental temperature; support with oxygen; a respirator or a ventilator; peripheral blood transfusion; iron supplementation; intravenous feeding; phototherapy; surgery; agents for the treatment of apnea (e.g., aminophylline, caffeine or doxapram, and the like); agents for the treatment of ARDS or RDS (e.g., a surfactant drug such as, e.g., CUROSURF® (Poractant Alfa; Douglas Pharmaceuticals)); antibiotics or antiviral drugs, anti-inflammatory agents (e.g., steroidal anti-inflammatory compounds, non-steroidal anti-inflammatory (NSAID) compounds), nitric oxide; antihistamines, immune suppressants, immunomodulatory compound (e.g., a TNF-α inhibitor); laser treatment (to treat, e.g., retinopathy of prematurity); etc. The treatment of infants (e.g., premature or full-term infants) is well-known in the art, and persons of skill in the art are able to select particular therapies, suitable for use in combination with administration of cord blood or cells obtained therefrom (e.g., total nucleated cells from umbilical cord blood) and/or cells obtained from placental perfusate (e.g., total nucleated cells from placental perfusate) or placental stem cells, on a case-by-case basis.

### 5.2 Umbilical Cord Blood

Umbilical cord blood may be collected in any medically or pharmaceutically-acceptable manner. Various methods for the collection of cord blood have been described. See, e.g., U.S. Pat. No. 6,102,871; U.S. Pat. No. 6,179,819; and U.S. Pat. No. 7,147,626, the contents of each of which are incorporated by reference in its entirety. The conventional technique for the collection of cord blood is based on the use of a needle or cannula, which is used with the aid of gravity to drain the cord blood from the placenta. See e.g., U.S. Pat. Nos. 5,192,553; 5,004,681; 5,372,581, and 5,415,665. Usually the needle or cannula is placed in the umbilical vein and the placenta is gently massaged to aid in draining the cord blood from the placenta. Cord blood may be collected into, for example, blood bags, transfer bags, or sterile plastic tubes.

In some embodiments, umbilical cord blood is obtained from a commercial cord blood bank (e.g., LifeBankUSA, etc.). In another embodiments, umbilical cord blood is collected from a post-partum mammalian placenta and used immediately (e.g., within 1, 2, 3, 4, 5, 6, 7, 8, 9, 10, 11 or 12 hours of collection). In other embodiments, the cord blood used to treat an infant (e.g., premature or full-term infant) is cord blood that has been cryopreserved. Umbilical cord blood can be collected from a single placenta or from a plurality of placentas.

Umbilical cord blood or cells obtained therefrom (e.g., total nucleated cells from umbilical cord blood) that is administered to an infant (e.g., premature or full-term infant) in accordance with the methods described herein can be autologous or heterologous. In particular embodiments, umbilical cord blood is obtained from the placenta of the infant (e.g., premature or full-term infant) to be treated. In certain embodiments, umbilical cord blood is obtained from a post-partum mammalian placenta of a full-term birth. In other embodiments, umbilical cord blood is obtained from a post-partum mammalian placenta of a premature birth, e.g., the placenta of a premature infant to be treated. In some embodiments, the placenta is the placenta of an infant born at about 23 to about 25 weeks of gestation. In some embodiments, embodiments, the placenta is the placenta of an infant born at about 26 to about 29 weeks of gestation. In some embodiments, embodiments, the placenta is the placenta of an infant born at about 30 to about 33 weeks of gestation. In some embodiments, embodiments, the placenta is the placenta of an infant born at about 34 to about 37 weeks of gestation. In some embodiments, the placenta is the placenta of an infant born at about 37 to about 42 weeks of gestation.

Cord blood, or total nucleated cells or stem cells derived therefrom, may be stored as collected from a single individual (i.e., as a single unit) for administration, or may be pooled with other units. Where umbilical cord blood is pooled from a plurality of placentas, the pooled cord blood can comprise umbilical cord blood from full-term births only, cord blood from a combination of full-term births, or cord blood from premature births only. For example, cord blood from the placenta of a premature infant can be combined with, e.g., cord blood from other premature infants, cord blood from full-term births only, or a combination of cord blood from both premature and full-term placentas. Cord blood, including autologous or allogeneic cord blood, can also be combined with peripheral blood. In certain embodiments, cord blood from premature births is used, as such cord blood comprises relatively high numbers of CD34⁺ stem cells per unit volume, compared to cord blood from full-term births. In certain embodiments, a unit of cord blood contains a sufficient number of cells such that at least about 1.5×10⁷, 2.0×10⁷, 2.5×10⁷, 3.0×10⁷, 3.5×10⁷, 4.0×10⁷, 4.5×10⁷, 5.0×10⁷, 5.5×10⁷, or 6.0×10⁷ cells obtained from said cord blood, e.g., total nucleated cells from cord blood, per kilogram body weight of an infant (e.g., premature infant or full-term infant) are administered.

In some embodiments, the total nucleated cells present in the cord blood comprises at least 5%, 10%, 15%, 20%, or more of CD38⁺CD45⁺ cells. In additional embodiments, the total nucleated cells present in the cord blood comprises at least 25%, 30%, 40%, 50%, or more of CD38⁻CD45⁻ cells. In some embodiments, the cord blood is prepared from preterm placenta. In other embodiments, the cord blood is prepared from full term placenta.

In specific embodiments, wherein the methods of treatment of infants described herein comprise the administration of cord blood or cells obtained therefrom (e.g., total nucleated cells from umbilical cord blood), said cord blood is autologous to the infant being treated.

### 5.3 Cells Obtained from Placental Perfusate

In certain embodiments, the cells used in the methods provided herein are cells obtained from placental perfusate.

Cells obtained from placental perfusate comprise total nucleated cells obtained from, e.g., isolated from, placental perfusate, a subset of nucleated cells obtained from placental perfusate, or cells cultured or proliferated from cells obtained directly from placental perfusate. In certain embodiments, cells obtained from placental perfusate comprise total nucleated cells from placental perfusate. In certain embodiments, the cells obtained from placental perfusate are obtained from a single placenta. In certain embodiments, the cells obtained from placental perfusate are obtained from more than one placenta. In certain embodiments, the cells obtained from placental perfusate are obtained from two placentas. As described herein, placental perfusate may be obtained from a placenta that has been drained of cord blood and perfused to remove residual blood, prior to perfusion to obtain placental cells. Placental perfusate may be obtained from a placenta that has been drained of cord blood but not perfused to remove residual blood. Placental perfusate may be obtained from a placenta that has neither been drained of cord blood nor perfused to remove residual blood. In the latter two embodiments, the placental cells, e.g., nucleated cells from placental perfusate, for example, total nucleated cells from placental perfusate, comprise nucleated cells from placental blood and/or cord blood. In a specific embodiment, placental perfusate used in the methods described herein is free of umbilical cord blood. In another specific embodiment, placental perfusate used in the methods described herein is substantially free of umbilical cord blood, e.g., said placental perfusate comprises less than 10%, less than 5%, less than 1%, less than 0.5%, or less than 0.1% cord blood.

Placental perfusate may be stored as collected from a single individual (i.e., as a single unit) for administration, or may be pooled with other units, e.g., from the same individual or from one or more other individuals. In certain embodiments, a unit of placental perfusate contains a sufficient number of cells such that at least about 0.5×10⁶, 1.0×10⁶, 1.5×10⁶, 2.0×10⁶, 2.5×10⁶, or 3.0×10⁶ cells obtained from placental perfusate, e.g., total nucleated cells, per kilogram body weight of an infant (e.g., premature infant or full-term infant) are administered.

Placentas for obtaining placental perfusate can be recovered following successful birth and placental expulsion. In certain embodiments, the placenta is from a full-term birth. In certain embodiments, the placenta is from a premature birth. In some embodiments, embodiments, the placenta is the placenta of an infant born at about 23 to about 25 weeks of gestation. In some embodiments, embodiments, the placenta is the placenta of an infant born at about 26 to about 29 weeks of gestation. In some embodiments, embodiments, the placenta is the placenta of an infant born at about 30 to about 33 weeks of gestation. In some embodiments, embodiments, the placenta is the placenta of an infant born at about 34 to about 37 weeks of gestation. In some embodiments, embodiments, the placenta is the placenta of an infant born at about 37 to about 42 weeks of gestation.

Cells obtained from placental perfusate (e.g., total nucleated cells from placental perfusate) can be autologous or allogeneic to the particular infant (e.g., premature or full-term infant) to be treated. In particular embodiments, cells obtained from placental perfusate (e.g., total nucleated cells from placental perfusate) are obtained from the placenta of the infant (e.g., premature or full-term infant) to be treated.

Cells obtained from placental perfusate (e.g., total nucleated cells from placental perfusate) used in the methods described herein can be obtained by any method. Placental perfusate can be obtained, e.g., as disclosed in U.S. Pat. No. 7,045,148, U.S. Pat. No. 7,255,879, and/or U.S. Pat. No. 8,057,788, the contents of each of which are incorporated herein by reference in their entirety. Such perfusion can, e.g., be perfusion by the pan method, wherein perfusion liquid is forced through the placental vasculature and perfusion fluid that exudes from the placenta, typically the maternal side, is collected in a pan containing the placenta. Perfusion can also, e.g., be a closed-circuit perfusion, wherein perfusion fluid is passed through, and collected from, only the fetal vasculature of the placenta. See, e.g., U.S. Pat. No. 8,057,788, the contents of which are incorporated herein by reference in their entirety. In a specific embodiment, such perfusion can be continuous, that is, perfusion fluid that has been passed through the placenta is passed through a second time, or a plurality of times, prior to isolation of cells obtained from placental perfusate (e.g., total nucleated cells from placental perfusate).

In one embodiment, cells obtained from placental perfusate (e.g., total nucleated cells from placental perfusate) comprise cells that are CD34⁺CD45⁻, CD34⁺CD45⁺, CD105⁺CD200⁺, or CD34⁺CD45⁺CD41⁺CD61⁺. Thus, in one embodiment, provided herein are methods of treating an infant (e.g., premature or full-term infant) comprising administering to the infant (e.g., premature or full-term infant) umbilical cord blood or cells obtained from umbilical cord blood (e.g., total nucleated cells from umbilical cord blood) and cells obtained from placental perfusate (e.g., total nucleated cells from placental perfusate). In another embodiment, provided herein is a method of treating an infant (e.g., premature or full-term infant) comprising administering to the infant (e.g., premature or full-term infant) cells obtained from placental perfusate (e.g., total nucleated cells from placental perfusate). In a specific embodiment, said cells obtained from placental perfusate (e.g., total nucleated cells from placental perfusate) comprise cells that are CD34⁺CD45⁻. In a specific embodiment, said cells obtained from placental perfusate (e.g., total nucleated cells from placental perfusate) comprise cells that are CD34⁺CD45⁺. In a specific embodiment, said cells obtained from placental perfusate (e.g., total nucleated cells from placental perfusate) comprise cells that are CD105⁺CD200⁺. In a specific embodiment, said cells obtained from placental perfusate (e.g., total nucleated cells from placental perfusate) comprise cells that are CD34⁺CD45⁺CD41⁺CD61⁺.

In another embodiment, the cells obtained from placental perfusate (e.g., total nucleated cells from placental perfusate) are homogenous, and sterile. In another specific embodiment, the cells obtained from placental perfusate (e.g., total nucleated cells from placental perfusate) are present in a pharmaceutical grade solution suitable for administration to a human.

Markers, such as cell surface markers, can be routinely determined according to methods well known in the art, e.g. by flow cytometry or fluorescence-activated cell sorting (FACS) analysis by washing and staining with an anti-cell surface marker antibody labeled with an appropriate fluorophore. For example, to determine the presence of CD34, cells may be washed in PBS and then double-stained with anti-CD34 phycoerythrin. The cells would then be analyzed using a standard flow cytometer. Alternatively, intracellular markers can also be examined via standard methodology.

In specific embodiments, wherein the methods of treatment of infants described herein comprise the administration of cells obtained from placental perfusate (e.g., total nucleated cells from placental perfusate), said cells obtained from placental are autologous to the infant being treated.

### 5.4 Placental Stem Cells

As used herein, the term “placental stem cell” refers to a stem cell, e.g., a tissue culture plastic-adherent stem cell (e.g., a multipotent cell), that is obtained from or derived from a mammalian placenta, or a portion thereof (e.g., amnion, chorion, and the like) regardless of morphology, cell surface markers, etc. The phrase encompasses a stem cell obtained directly from a placenta, e.g., as part of a population of placental cells in placental perfusate or digested placental tissue (digestate), or a stem cell that is part of a population of placental cells that has been expanded and/or passaged one or more times. The term does not, however, encompass stem cells derived solely from another tissue, e.g., placental blood or umbilical cord blood. The placenta comprises stem cell populations having, and distinguishable from each other by, for example, distinct sets of markers. Placental stem cells, and methods of obtaining the same, are described in detail in U.S. Pat. Nos. 7,045,148; 7,255,879; and in U.S. Pat. No. 8,057,788, the disclosures of which are hereby incorporated by reference in their entireties.

Placental stem cells can be recovered following successful birth and placental expulsion, resulting in the recovery of as many as one billion nucleated cells, which yield 50-100 million multipotent and pluripotent stem cells.

Placental stem cells useful in the methods and compositions described herein include, for example, pluripotent cells, multipotent cells, committed progenitor cells, hematopoietic progenitor cells, and mesenchymal-like stem cells from placenta. In one embodiment, the placental stem cells are contained within, or are derived from, placental perfusate. In other embodiments, placental stem cells are contained within, or are derived from, placental tissue that has been digested with one or more tissue-digesting enzymes (e.g., collagenase, hyaluronidase, and the like).

Placenta-derived stem cells used in the methods described herein can be derived from a single placenta, or from a plurality of placentas.

In certain embodiments, placental stem cells are obtained from a placenta of a full-term birth. In certain embodiments, the placental stem cells are obtained from a placenta of a premature birth. In some embodiments, embodiments, the placenta is the placenta of an infant born at about 23 to about 25 weeks of gestation. In some embodiments, embodiments, the placenta is the placenta of an infant born at about 26 to about 29 weeks of gestation. In some embodiments, embodiments, the placenta is the placenta of an infant born at about 30 to about 33 weeks of gestation. In some embodiments, embodiments, the placenta is the placenta of an infant born at about 34 to about 37 weeks of gestation. In some embodiments, embodiments, the placenta is the placenta of an infant born at about 37 to about 42 weeks of gestation.

Placental stem cells can be autologous or allogeneic to the particular infant (e.g., premature or full-term infant) to be treated. In particular embodiments, placental stem cells are obtained from the placenta of the infant (e.g., premature or full-term infant) to be treated.

Placental stem cells used in the methods described herein can be obtained by any method. Placental stem cells can be obtained by, for example, perfusion, as disclosed in U.S. Pat. No. 7,045,148, U.S. Pat. No. 7,255,879, and/or U.S. Pat. No. 8,057,788, the contents of each of which are incorporated herein by reference in their entirety. Such perfusion can, e.g., be perfusion by the pan method, wherein perfusion liquid is forced through the placental vasculature and perfusion fluid that exudes from the placenta, typically the maternal side, is collected in a pan containing the placenta. Perfusion can also, e.g., be a closed-circuit perfusion, wherein perfusion fluid is passed through, and collected from, only the fetal vasculature of the placenta. See, e.g., U.S. Pat. No. 8,057,788, the contents of which are incorporated herein by reference in their entirety. In a specific embodiment, such perfusion can be continuous, that is, perfusion fluid that has been passed through the placenta, and which comprises a plurality of placental cells, is passed through a second time, or a plurality of times, prior to isolation of placental cells.

Placenta-derived stem cells may also be obtained by physical or enzymatic disruption of the placenta using, e.g., proteases and/or other tissue-disruptive enzymes to disrupt the multicellular structure of the placenta. Such proteases may include neutral proteases or metalloproteases, e.g., collagenase, dispase, trypsin, elastase, and the like. Placental stem cells may also be obtained by physical disruption of the placenta using, e.g., mucolytic enzymes, for example, hyaluronidase.

The isolated perfused placenta used to provide cells for use in the methods described herein provides a source of large quantities of stem cells, e.g., cell populations enriched for CD34 stem cells, e.g., CD34⁺CD38⁻ or CD34⁺CD45⁻ stem cells, or CD34⁻ stem cells, e.g., CD34⁻CD38⁺ stem cells. The first collection of blood from the placenta is referred to as cord blood, which contains predominantly CD34⁺CD38⁺ hematopoietic progenitor cells. Within the first twenty-four hours of post-partum perfusion, CD34⁺CD38⁻ hematopoietic progenitor cells may be isolated from the placenta, along with CD34⁻CD38⁺ cells. After about twenty-four hours of perfusion, CD34⁻CD38⁻ cells can be isolated from the placenta along with the aforementioned cells. An isolated placenta that has been perfused for twenty-four hours or more provides a source of large quantities of stem cells enriched for CD34⁻CD38⁻ stem cells. In certain embodiments, the placental stem cells used in the methods described herein comprise placental stem cells isolated as placental perfusate, e.g., placental perfusate obtained within the first twenty-four hours of post-partum perfusion or after about twenty-four hours of perfusion.

At least one class of human placental stem cells has characteristics of embryonic stem or germ cells. For example, stem cells of this class are SSEA3⁻ (stage-specific embryonic antigen 3), SSEA4⁻, OCT-4⁺ (a stem cell transcription factor) and/or ABC-p⁺ (ATP-binding cassette (ABC) transporter protein), a marker profile exhibited by pluripotent stem cells that have not yet undergone differentiation. Thus, in one embodiment, placental stem cells useful in the methods described herein are SSEA3⁻, SSEA4⁻, OCT-4⁺ and/or ABC-p⁺. In another embodiment, the stem cells are OCT-4⁺ and ABC-p⁺. In another embodiment, the human placental stem cells do not express MHC Class 2 antigens. In yet another embodiment, the human placental stem cells are HLA-ABC+.

Placental stem cells usable in the methods described herein are CD10⁺, CD38⁻, CD29⁺, CD34⁻, CD44⁺, CD45⁻, CD54⁺, CD90⁺, SH2⁺, SH3⁺, SH4⁺, SSEA3⁻, SSEA4⁻, OCT-4⁺, and/or ABC-p. In a specific embodiment, the placental stem cells used in the methods described herein are CD10⁺, CD105⁺, CD200⁺, CD34⁻ placental stem cells.

In one embodiment, stem cells to be used in accordance with the methods described herein are CD34⁻, OCT-4⁺, CD73⁺, CD105⁺ and CD200⁺. In another embodiment, stem cells to be used in accordance with the methods described herein are CD34⁻, OCT-4⁺, CD73⁺, CD105⁺, CD200⁺ and HLA-G⁺. In certain embodiments, the placental stem cells comprise CD34⁻ cells. In other embodiments, the placental stem cells comprise OCT-4 cells. In other embodiments, the placental stem cells comprise cells that are CD73⁺, CD105⁺ and CD200⁺. In other embodiments, the placental stem cells comprise cells that are CD200⁺ or HLA-G⁺. In other embodiments, said placental stem cells comprise cells that are CD200⁺ and OCT-4⁺. In other embodiments, the placental stem cells comprise cells that are CD73⁺ and CD105⁺ and that facilitate the formation of one or more embryoid-like bodies in a population of placental cells comprising said cells when said population is cultured under conditions that allow the formation of an embryoid-like body. In other embodiments, the placental stem cells comprise cells that are CD73⁺, CD105⁺ and HLA-G⁺. In other embodiments, the placental stem cells comprise cells that are OCT-4⁺ and that facilitate the formation of one or more embryoid-like bodies in a population of placental cells comprising the stem cell when said population is cultured under conditions that allow formation of embryoid-like bodies.

Thus, in one embodiment, provided herein are methods of treating an infant (e.g., premature or full-term infant) comprising administering to the infant (e.g., premature or full-term infant) cord blood and placental stem cells. In another embodiment, provided herein is a method of treating an infant (e.g., premature or full-term infant) comprising administering to the infant (e.g., premature or full-term infant) placental stem cells. In a specific embodiment, said placental stem cells are placental stem cells contained within, or derived from, placental perfusate. In another specific embodiment, said placental stem cells are CD10⁺, CD105⁺, CD200⁺, CD34⁻ placental stem cells. In another specific embodiment, said stem cells are CD200⁺ or HLA-G⁺. In a specific embodiment, the stem cells are also CD200⁺ and HLA-G⁺. In a more specific embodiment, said stem cells are also CD73⁺ and CD105⁺. In another more specific embodiment, said stem cells are also CD34⁻, CD38⁻ or CD45⁻. In another more specific embodiment, said stem cells are also CD34⁻, CD38⁻ and CD45⁻. In another more specific embodiment, said stem cells are also CD34⁻, CD38⁻, CD45⁻, CD73⁺ and CD105⁺. In another specific embodiment, said CD200 or HLA-G⁺ stem cells facilitate the formation of embryoid-like bodies in a population of placenta-derived cells comprising the stem cells, under conditions that allow the formation of embryoid-like bodies.

In another specific embodiment, said placental stem cells are CD73⁺, CD105⁺ and CD200⁺. In a more specific embodiment, said stem cells are also HLA-G⁺. In another more specific embodiment, said stem cells are also CD34⁻, CD38⁻ or CD45⁻. In another more specific embodiment, said stem cells are also CD34⁻, CD38⁻ and CD45⁻. In a more specific embodiment, said stem cells are also CD34⁻, CD38⁻, CD45⁻, and HLA-G⁺. In another more specific embodiment, the CD73⁺, CD105⁺, and CD200⁺ stem cells facilitate the formation of one or more embryoid-like bodies in a population of placenta-derived cells comprising the stem cell, when the population is cultured under conditions that allow the formation of embryoid-like bodies.

In another specific embodiment, said placental stem cells are CD200⁺ and OCT-4⁺. In a more specific embodiment, the stem cells are also CD73⁺ and CD105⁺. In another more specific embodiment, said cells are also HLA-G⁺. In another specific embodiment, said stem cell is CD34⁻, CD38⁻ or CD45⁻. In another specific embodiment, said stem cell is CD34⁻, CD38⁻ and CD45⁻. In a more specific embodiment, said stem cell is CD34⁻, CD38⁻, CD45⁻, CD73⁺, CD105⁺ and HLA-G⁺. In another specific embodiment, the placental stem cells facilitate the production of one or more embryoid-like bodies by a population of placenta-derived cells that comprises the stem cells, when the population is cultured under conditions that allow the formation of embryoid-like bodies.

In another specific embodiment, said placental stem cells are CD73⁺, CD105⁺ and HLA-G⁺. In a more specific embodiment, said CD73⁺, CD105⁺ and HLA-G⁺ stem cells are CD34⁻, CD38⁻ or CD45⁻. In another more specific embodiment, said CD73⁺, CD105⁺ and HLA-G⁺ stem cells are OCT-4⁺. In another specific embodiment, said placental stem cells are CD34⁻, CD38⁻ and CD45⁻. In another specific embodiment, said placental stem cells are CD200⁺. In a more specific embodiment, said placental stem cell is CD34⁻, CD38⁻, CD45⁻, OCT-4⁺ and CD200⁺. In another more specific embodiment, said placental stem cell facilitates the formation of embryoid-like bodies in a population of placental cells comprising said stem cell, when the population is cultured under conditions that allow the formation of embryoid-like bodies.

In another specific embodiment, the placental stem cells can be one or more of SSEA3⁻, SSEA4⁻, OCT-4⁺ and ABC-p⁺. In another embodiment, the placental stem cells are OCT-4⁺ and ABC-p⁺. In one embodiment, the human placental stem cells do not express MHC Class II antigens. In other embodiments, the placental stem cells are one or more of CD10⁺, CD38⁻, CD29⁺, CD34⁻, CD44⁺, CD45⁻, CD54⁺, CD90⁺, SH2⁺, SH⁺, SH4⁺, SSEA3⁻, SSEA4⁻, OCT-4⁺, and/or ABC-p⁺.

In another embodiment, the placental stem cells are homogenous, and sterile. In another specific embodiment, the placental stem cells are present in a pharmaceutical grade solution suitable for administration to a human.

Markers, such as cell surface markers, can be routinely determined according to methods well known in the art, e.g. by flow cytometry or fluorescence-activated cell sorting (FACS) analysis by washing and staining with an anti-cell surface marker antibody labeled with an appropriate fluorophore. For example, to determine the presence of CD34 or CD38, cells may be washed in PBS and then double-stained with anti-CD34 phycoerythrin and anti-CD38 fluorescein isothiocyanate (Becton Dickinson, Mountain View, Calif.). The cells would then be analyzed using a standard flow cytometer. Alternatively, intracellular markers can also be examined via standard methodology.

In specific embodiments, wherein the methods of treatment of infants described herein comprise the administration of placental stem cells, said placental stem cells are autologous to the infant being treated.

### 5.5 Collection and Characterization of Cells

Umbilical cord blood and cells obtained therefrom, placental perfusate and cells obtained therefrom, and placental stem cells for use in the methods described herein can be collected and isolated by any technique known to those of skill in the art. In certain embodiments, umbilical cord blood and cells obtained therefrom, placental perfusate and cells obtained therefrom, or placental stem cells are isolated and collected as described in U.S. Pat. No. 7,045,148 or in U.S. Pat. No. 8,057,788, the contents of each of which are incorporated by reference in their entireties. Exemplary such methods are described below.

### 5.5.1 Perfusion

### 5.5.1.1 Pretreatment of Placenta

The collection of cord blood, placental perfusate, and placental stem cells starts with collection of a placenta, e.g., a human placenta. In certain embodiments, a human placenta is recovered shortly after its expulsion after birth and, in certain embodiments, the cord blood in the placenta is recovered. In specific embodiments, the placenta is subjected to a conventional cord blood recovery process as an adjunct to treatment of the premature infant, e.g., recovery of cord blood in response to the particular premature infant's need. Cord blood may also be obtained from a commercial cord blood banking service, e.g., LifeBankUSA, Cedar Knolls, N.J.

Typically, cord blood collection proceeds as follows. Postpartum (after either Caesarian delivery or natural birth), the placenta is exsanguinated, e.g., drained of cord blood. Prior to cord blood collection, the placenta may be stored under sterile conditions and at a temperature of, e.g., about 5° C. to about 25° C., or at about room temperature. In certain embodiments, the proximal umbilical cord is clamped, e.g., within 4-5 cm (centimeter) of the insertion into the placental disc prior to cord blood recovery. In other embodiments, the proximal umbilical cord is clamped after cord blood recovery but prior to further processing of the placenta. Conventional techniques for the collection of cord blood may be used. Typically a needle or cannula is used, with the aid of gravity, to drain cord blood from (i.e., exsanguinate) the placenta (see, e.g., Boyse et al., U.S. Pat. No. 5,192,553; Boyse et al., U.S. Pat. No. 5,004,681; Anderson, U.S. Pat. No. 5,372,581; Hessel et al., U.S. Pat. No. 5,415,665). The needle or cannula is usually placed in the umbilical vein and the placenta is gently massaged to aid in draining cord blood from the placenta.

The placenta may then be stored for a period of about 1 hour to about 72 hours or about 4 to about 24 hours, prior to perfusing the placenta to remove any residual cord blood.

After cord blood collection, the placenta can be stored in an anticoagulant solution at a temperature of about 5° C. to about 25° C., e.g., at about room temperature. Suitable anticoagulant solutions are well known in the art. For example, a solution of heparin or warfarin sodium can be used. In one embodiment, the anticoagulant solution comprises a solution of heparin (1% w/w in 1:1000 solution). The drained placenta can be stored for no more than 36 hours before placental stem cells are collected, as described below.

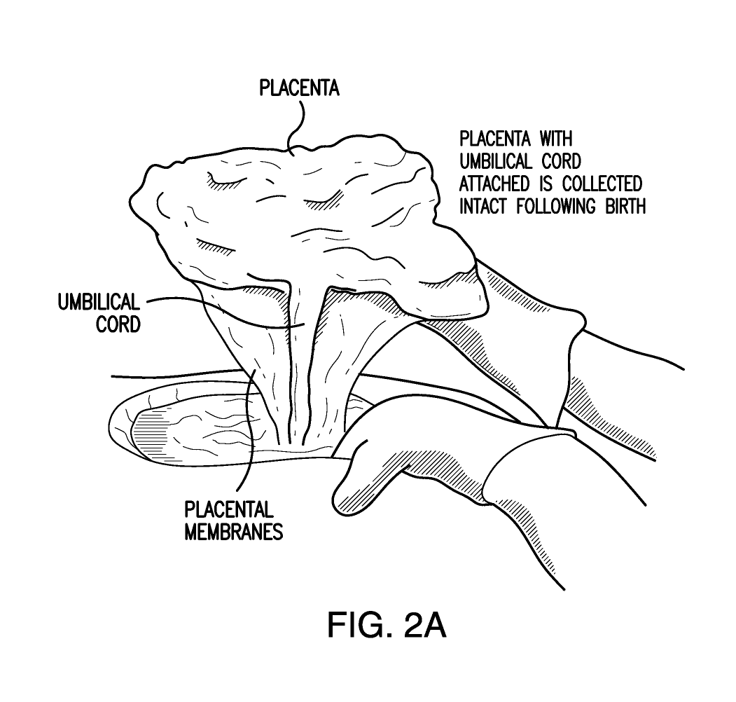

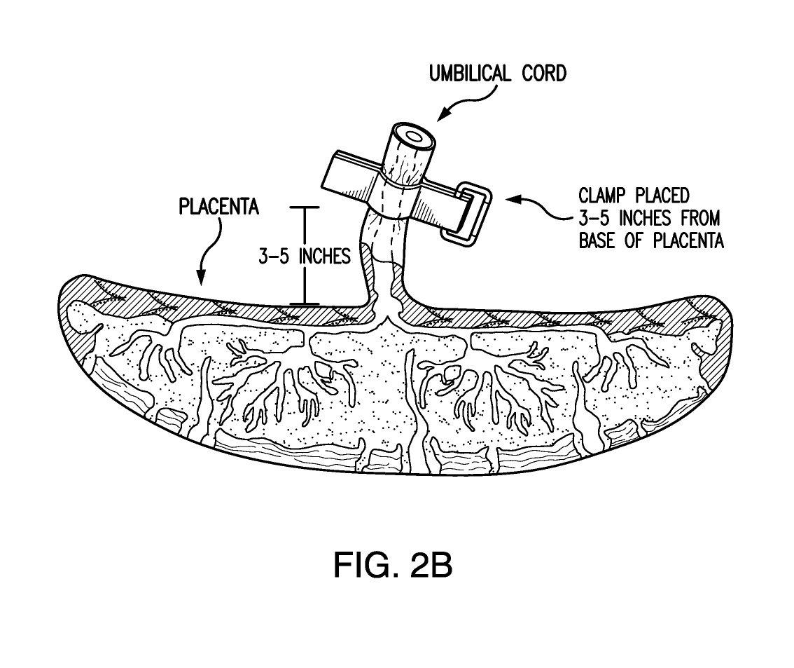

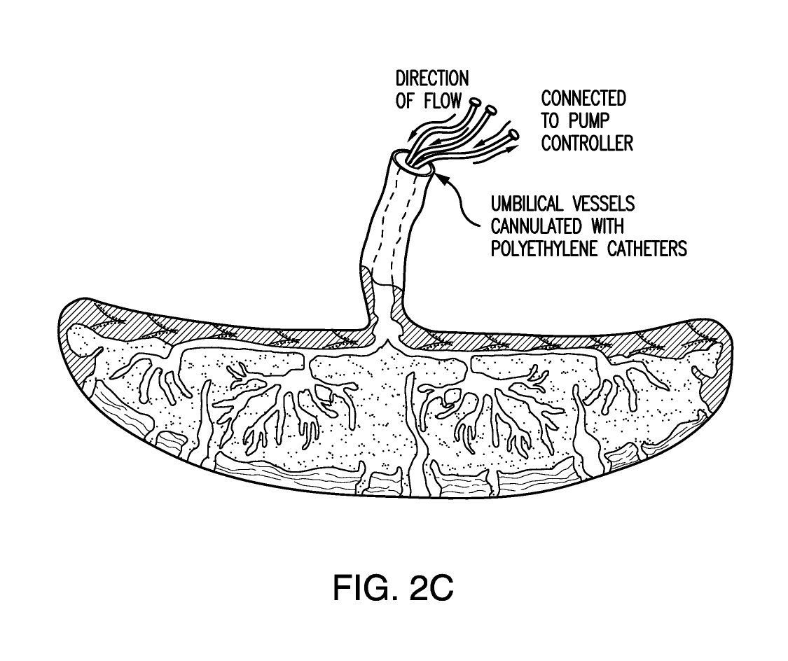

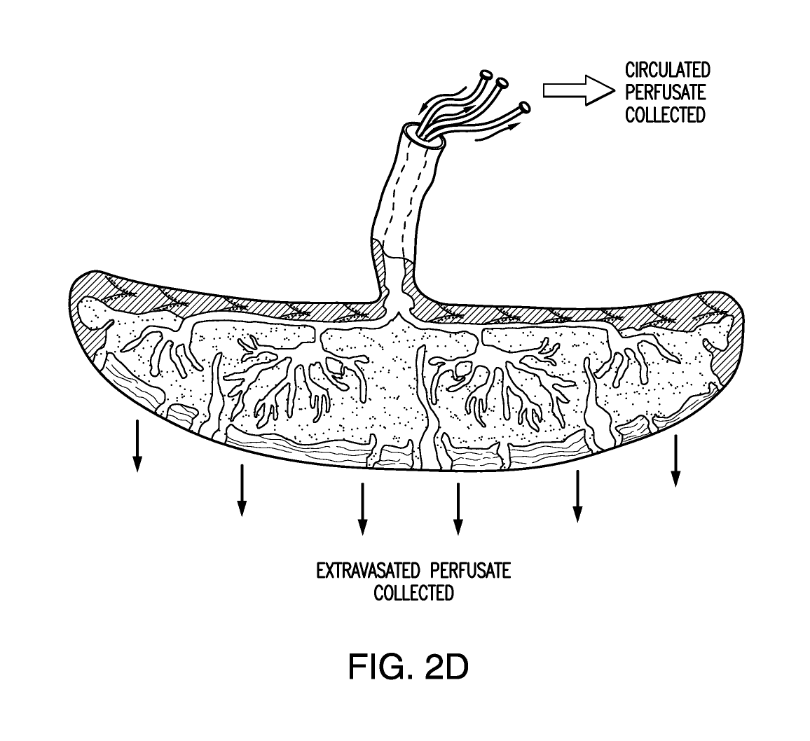

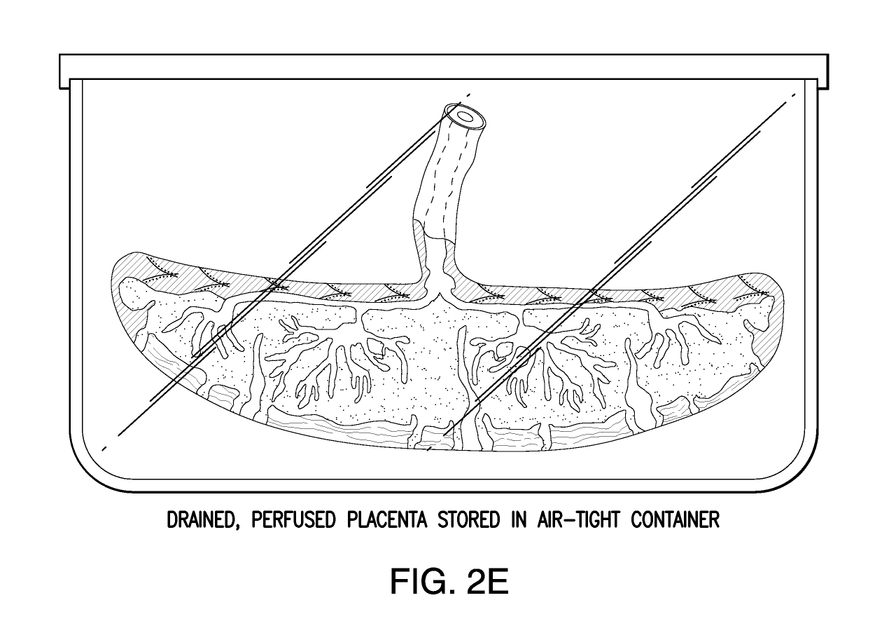

Typically, a placenta is transported from the delivery or birthing room to another location, e.g., a laboratory, for recovery of cord blood and/or drainage and stem cell collection by, e.g., perfusion or enzymatic digestion of placental tissue. The placenta can be transported in a sterile, thermally insulated transport device (maintaining the temperature of the placenta between, e.g., about 20° C. and about 28° C.), for example, by placing the placenta, with clamped proximal umbilical cord, in a sterile zip-lock plastic bag, which is then placed in an insulated container, as shown in FIGS. 2a-e.

In one embodiment, the placenta is recovered from a patient by informed consent and a complete medical history of the patient prior to, during and after pregnancy is also taken and is associated with the placenta. These medical records can be used to coordinate subsequent use of the placenta or the cells, e.g., cells obtained from placental perfusate or placental stem cells harvested therefrom. For example, such human cells obtained from placental perfusate or placental stem cells can then easily be used for personalized medicine for the infant (e.g., premature or full-term infant) to be treated.

### 5.5.1.2 Exsanguination of Placenta and Removal of Residual Cells

After cord blood recovery, cells obtained from placental perfusate or placental stem cells are recovered from the placenta by, e.g., perfusion. In one aspect, the exsanguinated placenta is perfused with a suitable aqueous perfusion fluid to remove residual cord blood. The perfusion solution can be any aqueous isotonic fluid. In one embodiment, an anticoagulant (e.g., heparin, warfarin sodium) is dissolved. Such aqueous isotonic fluids for perfusion are well known in the art, and include, e.g., nutrient media, saline solutions, e.g., phosphate buffered saline or a 0.9 N sodium chloride solution. When used, the perfusion fluid can comprise the anticoagulant at a concentration that is sufficient to prevent the formation of clots of any residual cord blood. In a specific embodiment, a concentration of from 1 to 100 units of heparin is employed. In another specific embodiment, a concentration of 1 to 10 units of heparin per ml is employed. In one embodiment, apoptosis inhibitors, such as free radical scavengers, in particular oxygen free radical scavengers, can be used during and immediately after exsanguination and then these agents can be washed from the placenta. In accordance with this embodiment, the isolated placenta may be stored under hypothermic conditions in order to prevent or inhibit apoptosis.

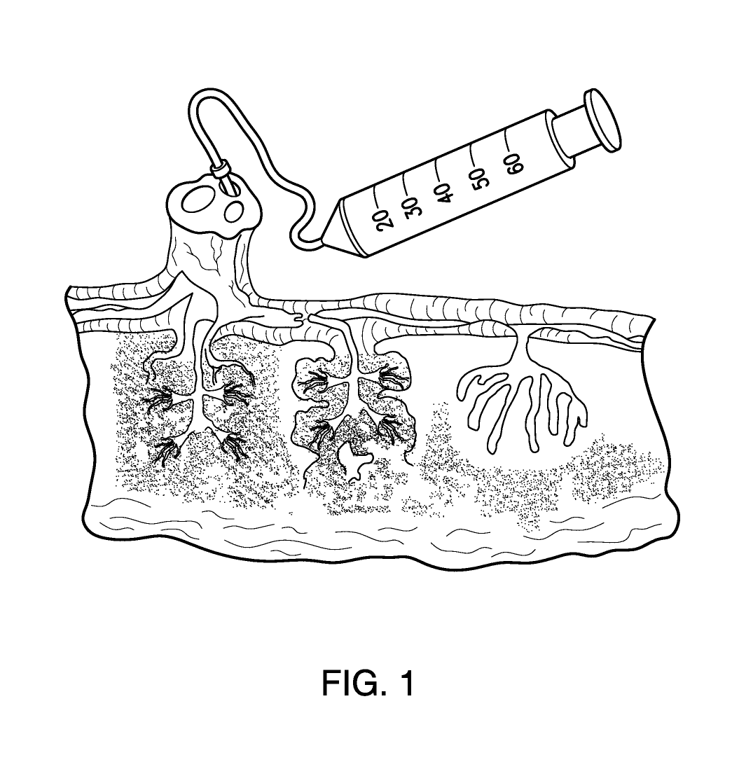

In one embodiment, prior to collection of cells obtained from placental perfusate or placental stem cells, the placenta is flushed with, e.g., 10-100 mL of perfusion fluid to remove substantially all remaining cord blood. Typically such flushing is performed by passage of the perfusion fluid through either or both of the umbilical artery and umbilical vein, using a gravity flow into the placenta. The placenta can be oriented (e.g., suspended) in such a manner that the umbilical artery and umbilical vein are located at the highest point of the placenta. In one embodiment, the umbilical artery and the umbilical vein are connected simultaneously, as shown in FIG. 1, to a pipette that is connected via a flexible connector to a reservoir of the perfusion fluid. The perfusion fluid is passed into the umbilical vein and artery and collected in a suitable open vessel from the surface of the placenta that was attached to the uterus of the mother during gestation. The perfusion fluid may also be introduced through the umbilical cord opening and allowed to flow or percolate out of openings in the wall of the placenta which interfaced with the maternal uterine wall.

In one embodiment, the proximal umbilical cord is clamped during perfusion, e.g., is clamped within 4-5 cm (centimeter) of the cord's insertion into the placental disc.

In one embodiment, a sufficient amount of perfusion fluid is used that will result in removal of essentially all residual cord blood and subsequent collection or recovery of placental cells, including but not limited to placental stem cells, e.g., embryonic-like stem cells and progenitor cells, that remain in the placenta after removal of the cord blood.

Generally from 30 to 100 ml (milliliter) of perfusion fluid is adequate to exsanguinate the placenta and to recover an initial population of cells, e.g., embryonic-like cells from the placenta, but more or less perfusion fluid may be used depending on the observed results.

In a specific embodiment, placental perfusate used in the methods described herein is free of umbilical cord blood. In another specific embodiment, placental perfusate used in the methods described herein is substantially free of umbilical cord blood, e.g., said placental perfusate comprises less than 10%, less than 5%, less than 1%, less than 0.5%, or less than 0.1% cord blood.

Cell types are then isolated from the collected perfusate by employing techniques known by those skilled in the art, such as for example, but not limited to density gradient centrifugation, magnetic cell separation, cell sorting by FACS, affinity cell separation or differential adhesion techniques.

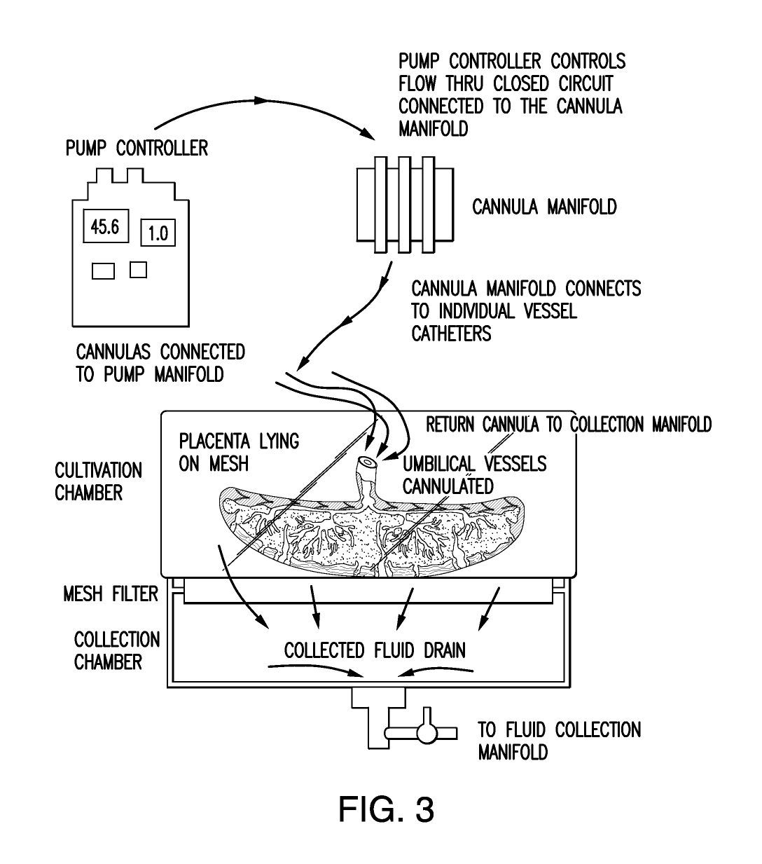

In one embodiment, a placenta is placed in a sterile basin and washed with 500 ml of phosphate-buffered normal saline. The wash fluid is then discarded. The umbilical vein is then cannulated with a cannula, e.g., a TEFLON® or plastic cannula, that is connected to a sterile connection apparatus, such as sterile tubing. The sterile connection apparatus is connected to a perfusion manifold, as shown in FIG. 3. The container containing the placenta is then covered and the placenta is maintained at room temperature (20-25° C.) for a desired period of time, e.g., from 2 to 24 hours, an, in certain embodiments, no longer than 48 hours. The placenta may be perfused continually, with equal volumes of perfusate introduced and effluent perfusate removed or collected. Alternatively, the placenta may be perfused periodically, e.g., at every 2 hours; at 4, 8, 12, and 24 hours; or at other intervals during culturing, with a volume of perfusate, e.g., 100 ml of perfusate (sterile normal saline supplemented with or without 1000 u/l heparin and/or EDTA and/or CPDA (creatine phosphate dextrose)). In the case of periodic perfusion, equal volumes of perfusate can be introduced and removed from the culture environment of the placenta, so that a stable volume of perfusate bathes the placenta at all times.

The effluent perfusate that escapes the placenta, e.g., at the opposite surface of the placenta, is collected and processed to isolate cells, e.g., embryonic-like stem cells, progenitor cells or other cells of interest.

The number and type of cells propagated may easily be monitored by measuring changes in morphology and cell surface markers using standard cell detection techniques such as flow cytometry, cell sorting, immunocytochemistry (e.g., staining with tissue specific or cell-marker specific antibodies), fluorescence activated cell sorting (FACS), magnetic activated cell sorting (MACS), by examination of the morphology of cells using light or confocal microscopy, or by measuring changes in gene expression using techniques well known in the art, such as PCR and gene expression profiling.

In one embodiment, total nucleated cells from placental perfusate are isolated by differential centrifugation in order to separate the total nucleated cells from, e.g., cell debris, serum, or enucleated cells.

In one embodiment, placental stem cells may be sorted using a fluorescence activated cell sorter (FACS). Fluorescence activated cell sorting (FACS) is a well-known method for separating particles, including cells, based on the fluorescent properties of the particles (Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In another embodiment, magnetic beads can be used to separate cells. The cells may be sorted using a magnetic activated cell sorting (MACS) technique, a method for separating particles based on their ability to bind magnetic beads (0.5-100 μm diameter). A variety of useful modifications can be performed on the magnetic microspheres, including covalent addition of antibody that specifically recognizes a cell-solid phase surface molecule or hapten. A magnetic field is then applied, to physically manipulate the selected beads. The beads are then mixed with the cells to allow binding. Cells are then passed through a magnetic field to separate out cells having cell surface markers. These cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

In certain embodiments, the drained, exsanguinated placenta is cultured as a bioreactor, i.e., an ex vivo system for propagating placental stem cells.

In certain embodiments, the placenta is cultured or cultivated under aseptic conditions in a container or other suitable vessel, and perfused with perfusate solution (e.g., a normal saline solution such as phosphate buffered saline (“PBS”), or, preferably, a 0.9 N saline solution) with or without an anticoagulant (e.g., heparin, warfarin sodium, coumarin, bishydroxycoumarin), and/or with or without an antimicrobial agent (e.g., B-mercaptoethanol (0.1 mM); antibiotics such as streptomycin (e.g., at 40-100 μg/ml), penicillin (e.g., at 40 U/ml), amphotericin B (e.g., at 0.5 μg/ml), or the like. Various media may be used as perfusion fluid for culturing or cultivating the placenta, such as DMEM, Ham's F-12, M199, RPMI, Fisher's, Iscove's, McCoy's and combinations thereof, supplemented with fetal bovine serum (FBS), whole human serum (WHS), or human umbilical cord serum collected at the time of delivery of the placenta. The same perfusion fluid used to exsanguinate the placenta of residual cord blood may be used to culture or cultivate the placenta, without the addition of anticoagulant agents.

The effluent perfusate comprises both circulated perfusate, which has flowed through the placental circulation, and extravasated perfusate, which exudes from or passes through the walls of the blood vessels into the surrounding tissues of the placenta. The effluent perfusate, or circulated perfusate, or, preferably, both the circulated and extravasated perfusates are collected, preferably in a sterile receptacle. Alterations in the conditions in which the placenta is maintained and the nature of the perfusate can be made to modulate the volume and composition of the effluent perfusate.

The number of propagated cells in the placental bioreactor can be maintained in a continuous state of balanced growth by periodically or continuously removing a portion of a culture medium or perfusion fluid that is introduced into the placental bioreactor, and from which the propagated cells may be recovered. Fresh medium or perfusion fluid is introduced at the same rate or in the same amount. To use the placenta as a bioreactor, it may be cultured for varying periods of time under sterile conditions by perfusion with perfusate solution as disclosed herein. In specific embodiments, the placenta is cultured for at least about 12, 24, 36, or 48 hours, or for 3-5 days, 6-10 days, or for one to two weeks. In one embodiment, the placenta is cultured for about 48 hours. The cultured placenta is can be “fed” periodically by the removal of spent media and the cells suspended in the media, and addition of fresh media. The cultured placenta can, e.g., be stored under sterile conditions to reduce the possibility of contamination, and maintained under intermittent and periodic pressurization to create conditions that maintain an adequate supply of nutrients to the cells of the placenta. Perfusion and culture of the placenta can be both automated and computerized for efficiency and increased capacity.

In certain embodiments, placental stem cells are induced to propagate in the placenta bioreactor by introduction of nutrients, hormones, vitamins, growth factors, or any combination thereof, into the perfusion solution. Serum and other growth factors may be added to the propagation perfusion solution or medium. Growth factors are usually proteins and include, but are not limited to: cytokines, lymphokines, interferons, colony stimulating factors (CSFs), interferons, chemokines, and interleukins. Other growth factors that may be used include recombinant human hematopoietic growth factors including ligands, stem cell factors, thrombopoietin (Tpo), granulocyte colony-stimulating factor (G-CSF), leukemia inhibitory factor, basic fibroblast growth factor, placenta derived growth factor and epidermal growth factor.

### 5.5.1.3 Collection of Cells from the Placenta

As disclosed above, after exsanguination and perfusion of the placenta, embryonic-like stem cells migrate into the drained, empty microcirculation where, according to methods described herein, they are collected, e.g., by collecting the effluent perfusate in a collecting vessel.

In specific embodiments, cells cultured in the placenta are isolated from the effluent perfusate using techniques known by those skilled in the art, such as, for example, density gradient centrifugation, magnetic cell separation, FACS sorting, or other cell separation or sorting methods well known in the art, and sorted.

In a specific embodiment, cells collected from the placenta can be recovered from the effluent perfusate by centrifugation at, e.g., about 5000×g for about 15 minutes at room temperature, which separates cells from contaminating debris and platelets. The cell pellets are resuspended in, e.g., IMDM serum-free medium containing 2 U/ml heparin and 2 mM EDTA (GibcoBRL, NY). The total mononuclear cell fraction can be isolated using apheresis, e.g., using a commercial collection kit such as LYMPHOPREP™ (Nycomed Pharma, Oslo, Norway). Cells are then counted using, e.g., a hemocytometer. Viability is typically evaluated by trypan blue exclusion. Isolation of cells can, e.g., be achieved by “differential trypsinization,” using a solution of, e.g., 0.05% trypsin with 0.2% EDTA (Sigma, St. Louis Mo.). Differential trypsinization is possible because fibroblastoid cells trypsinized in this manner detach from plastic cell culture surfaces within about five minutes, whereas other adherent populations require more than about 20-30 minutes incubation with trypsin. The detached fibroblastoid cells are harvested following trypsinization and trypsin neutralization, using Trypsin Neutralizing Solution (TNS, BioWhittaker). The cells can then be washed in a nutrient medium such as H.DMEM, and resuspended in, e.g. MSCGM.

In another embodiment, the isolated placenta is perfused for a period of time without collecting the perfusate, such that the placenta may be perfused for 2, 4, 6, 8, 10, 12, 20 or 24 hours or even days before the perfusate is collected. In such embodiments, for example, perfusion fluid can be introduced into the placenta and allowed to occupy the placental vasculature for a time prior to collection, or in the case of circulated perfusate, the perfusion fluid can be recirculated for such a time.

In another embodiment, cells cultured in the placenta bioreactor are isolated from the placenta by physically dissecting the cells away from the placenta.

In another embodiment, cells cultured in the placenta bioreactor are isolated from the placenta by dissociating the tissues of the placenta or a portion thereof, and recovering the cultured cells by art-known cell separation or sorting methods such as density gradient centrifugation, magnetic cell separation, FACS sorting, etc.

In one embodiment, perfusion of the placenta and collection of effluent perfusate is repeated once or twice during the culturing of the placenta, until the number of recovered nucleated cells falls below 100 cells/ml. The perfusates are pooled and subjected to light centrifugation to remove platelets, debris and de-nucleated cell membranes. The nucleated cells are then isolated by Ficoll-Hypaque density gradient centrifugation and after washing, resuspended in HDMEM. For isolation of the adherent cells, aliquots of 5-10×10⁶ cells are placed in each of several T-75 flasks and cultured with commercially available Mesenchymal Stem Cell Growth Medium (MSCGM) obtained from BioWhittaker, and placed in a tissue culture incubator (37° C., 5% CO₂). After 10 to 15 days, non-adherent cells are removed from the flasks by washing with PBS. The PBS is then replaced by MSCGM. Flasks can be examined daily for the presence of various adherent cell types and in particular, for identification and expansion of clusters of fibroblastoid cells.

In other embodiments, the cells collected from the placenta are cryopreserved for use at a later time. Methods for cryopreservation of cells, such as stem cells, are well known in the art, for example, cryopreservation using the methods of Boyse et al. (U.S. Pat. No. 5,192,553, issued Mar. 9, 1993) or Hu et al. (WO 00/73421, published Dec. 7, 2000).

### 5.5.2 Isolation of Placental Stem Cells by Physical Disruption of the Placenta

Placental stem cells can be collected from a mammalian placenta by physical disruption, e.g., enzymatic digestion, of the organ. For example, the placenta, or a portion thereof, may be, e.g., crushed, sheared, minced, diced, chopped, macerated or the like, while in contact with the stem cell collection composition, and the tissue subsequently digested with one or more enzymes. The placenta, or a portion thereof, may also be physically disrupted and digested with one or more enzymes, and the resulting material then immersed in, or mixed into, the stem cell collection composition. Any method of physical disruption can be used, provided that the method of disruption leaves a plurality, or a majority, or at least 60%, 70%, 80%, 90%, 95%, 98%, or 99% of the cells in said organ viable, as determined by, e.g., trypan blue exclusion.

The placenta can be dissected into components prior to physical disruption and/or enzymatic digestion and stem cell recovery. For example, placenta-derived stem cells can be obtained from the amniotic membrane, chorion, umbilical cord, placental cotyledons, or any combination thereof. Placenta-derived stem cells can, e.g., be obtained from placental tissue comprising amnion and chorion. Typically, placenta-derived stem cells can be obtained by disruption of a small block of placental tissue, e.g., a block of placental tissue that is about 1, 2, 3, 4, 5, 6, 7, 8, 9, 10, 20, 30, 40, 50, 60, 70, 80, 90, 100, 200, 300, 400, 500, 600, 700, 800, 900 or about 1000 cubic millimeters in volume.

One stem cell collection composition comprises one or more tissue-disruptive enzyme(s). Enzymatic digestion can use a combination of enzymes, e.g., a combination of a matrix metalloprotease and a neutral protease, for example, a combination of collagenase and dispase. In one embodiment, enzymatic digestion of placental tissue uses a combination of a matrix metalloprotease, a neutral protease, and a mucolytic enzyme for digestion of hyaluronic acid, such as a combination of collagenase, dispase, and hyaluronidase or a combination of LIBERASE® (Boehringer Mannheim Corp., Indianapolis, Ind.) and hyaluronidase. Other enzymes that can be used to disrupt placenta tissue include papain, deoxyribonucleases, serine proteases, such as trypsin, chymotrypsin, or elastase. Serine proteases may be inhibited by alpha 2 microglobulin in serum and therefore the medium used for digestion is usually serum-free. EDTA and DNase are commonly used in enzyme digestion procedures to increase the efficiency of cell recovery. The digestate can be diluted so as to avoid trapping stem cells within the viscous digest.

Any combination of tissue digestion enzymes can be used. Typical concentrations for tissue digestion enzymes include, e.g., 50-200 U/mL for collagenase I and collagenase IV, 1-10 U/mL for dispase, and 10-100 U/mL for elastase. Proteases can be used in combination, that is, two or more proteases in the same digestion reaction, or can be used sequentially in order to liberate placental stem cells. For example, in one embodiment, a placenta, or part thereof, is digested first with an appropriate amount of collagenase I at 2 mg/ml for 30 minutes, followed by digestion with trypsin, 0.25%, for 10 minutes, at 37° C. Serine proteases can be used consecutively following use of other enzymes.

In another embodiment, the tissue can further be disrupted by the addition of a chelator, e.g., ethylene glycol bis(2-aminoethyl ether)-N,N,N′N′-tetraacetic acid (EGTA) or ethylenediaminetetraacetic acid (EDTA) to the stem cell collection composition comprising the stem cells, or to a solution in which the tissue is disrupted and/or digested prior to isolation of the stem cells with the stem cell collection composition.

It will be appreciated that where an entire placenta, or portion of a placenta comprising both fetal and maternal cells (for example, where the portion of the placenta comprises the chorion or cotyledons), the placenta-derived stem cells collected will comprise a mix of placenta-derived stem cells derived from both fetal and maternal sources. Where a portion of the placenta that comprises no, or a negligible number of, maternal cells (for example, amnion), the placenta-derived stem cells collected will comprise almost exclusively fetal placenta-derived stem cells.

Placental stem cells in the digested placental tissue can be isolated by, e.g., culturing of such cells with other cells in the digested tissue and isolating by differential trypsinization as described elsewhere herein. Alternatively, such cells can be purified using one or more antibodies to placental stem cell markers, followed by, e.g., magnetic bead separation. See e.g. Section 5.5.1.2.

### 5.6 Combinations of Umbilical Cord Blood or Cells Obtained Therefrom and Cells Obtained from Placental Perfusate or Placental Stem Cells

In certain embodiments, provided herein is a method of treating a disorder or condition in an infant, e.g., a disease or disorder caused by or associated with premature birth by using a combination of umbilical cord blood or cells obtained therefrom (e.g., total nucleated cells from umbilical cord blood) and cells obtained from placental perfusate (e.g., total nucleated cells from placental perfusate) or placental stem cells. The placental stem cells can be stem cells contained within placental perfusate; placental stem cells initially collected from placental perfusate; placental stem cells collected from digestion of placental tissue; placental stem cells from placental perfusate or digestion of placental tissue; and/or tissue culture plate adherent placental stem cells, wherein the placental stem cells have been cultured in cell culture for a time sufficient for the placental stem cells to propagate for, e.g., about 1, 2, 3, 4, 5, 6, 7, 8, 9, 10, 11, 12, 13, 14, 15, 16, 17, 18, 19, 20, 22, 24, 26, 28, 30, 32, 34, 36, 38 or 40 population doublings; or any combination of the foregoing. Placental stem cells to be combined with cord blood or cells obtained therefrom (e.g., total nucleated cells from umbilical cord blood) in the treatment of infants (e.g., premature or full-term infants) can be from a single placenta or a plurality of placentas.

The ratio of umbilical cord blood or cells obtained therefrom (e.g., total nucleated cells from umbilical cord blood) and cells obtained from placental perfusate (e.g., total nucleated cells from placental perfusate) or placental stem cells can be determined according to the judgment of those of skill in the art. In certain embodiments, the ratio of umbilical cord blood or cells obtained therefrom (e.g., total nucleated cells from umbilical cord blood) to cells obtained from placental perfusate (e.g., total nucleated cells from placental perfusate) or placental stem cells is about 100,000,000:1, 50,000,000:1, 20,000,000:1, 10,000,000:1, 5,000,000:1, 2,000,000:1, 1,000,000:1, 500,000:1, 200,000:1, 100,000:1, 50,000:1, 20,000:1, 10,000:1, 5,000:1, 2,000:1, 1,000:1, 500:1, 200:1, 100:1, 50:1, 20:1, 10:1, 5:1, 2:1, 1:1; 1:2; 1:5; 1:10; 1:100; 1:200; 1:500; 1:1,000; 1:2,000; 1:5,000; 1:10,000; 1:20,000; 1:50,000; 1:100,000; 1:500,000; 1:1,000,000; 1:2,000,000; 1:5,000,000; 1:10,000,000; 1:20,000,000; 1:50,000,000; or about 1:100,000,000, comparing numbers of total nucleated cells in each population, or comparing total numbers of stem cells in each population. In certain embodiments, the ratio of umbilical cord blood or cells obtained therefrom (e.g., total nucleated cells from umbilical cord blood) to cells obtained from placental perfusate (e.g., total nucleated cells from placental perfusate) or placental stem cells is between about 20:1 and about 1:20, or is about 1:10, about 1:5, about 1:1, about 5:1 or about 10:1.

Cells obtained from placental perfusate (e.g., total nucleated cells from placental perfusate), placental stem cells, cord blood, and/or cells obtained from cord blood (e.g., total nucleated cells from umbilical cord blood) can be combined prior to administration to an infant (e.g., premature or full-term infant), or can be administered separately.

### 5.6.1 Pharmaceutical Compositions

Also encompassed herein are pharmaceutical compositions comprising umbilical cord blood or cells obtained therefrom (e.g., total nucleated cells from umbilical cord blood) and cells obtained from placental perfusate (e.g., total nucleated cells from placental perfusate) or placental stem cells, and a pharmaceutically-acceptable carrier. In accordance with this embodiment, the combined umbilical cord blood or cells obtained therefrom (e.g., total nucleated cells from umbilical cord blood) and cells obtained from placental perfusate (e.g., total nucleated cells from placental perfusate) or placental stem cells described herein may, e.g., be formulated as an injectable composition (e.g., WO 96/39101, incorporated herein by reference in its entirety). In another embodiment, the combined umbilical cord blood or cells obtained therefrom (e.g., total nucleated cells from umbilical cord blood) and cells obtained from placental perfusate (e.g., total nucleated cells from placental perfusate) or placental stem cells described herein may be formulated using polymerizable or cross linking hydrogels as described, e.g., in U.S. Pat. Nos. 5,709,854; 5,516,532; 5,654,381.

In another embodiment, provided for herein is the maintenance of each population of the combined umbilical cord blood or cells obtained therefrom (e.g., total nucleated cells from umbilical cord blood) and cells obtained from placental perfusate (e.g., total nucleated cells from placental perfusate) or placental stem cells, prior to administration to an individual, as separate pharmaceutical compositions to be administered sequentially or jointly to create the combined stem cell population in vivo. Each component may be stored and/or used in a separate container, e.g., a single bag (e.g., blood storage bag from Baxter, Becton-Dickinson, Medcep, National Hospital Products, Terumo, etc.) or separate syringe, which contains a single type of cell or cell population. In a specific embodiment, cord blood, or cord blood-derived nucleated or stem cells, are contained in one bag, and placental perfusate, cells obtained therefrom, or placental stem cells from placental perfusate, are contained in a second bag.

A population of cells obtained from placental perfusate (e.g., total nucleated cells from placental perfusate) can be enriched. In a specific embodiment, the population of cells comprising total nucleated cells from placental perfusate is enriched by removal of red blood cells and/or granulocytes according to standard methods. A population of placental stem cells can be enriched. In a specific embodiment, a population of cells comprising placental stem cells is enriched by removal of red blood cells and/or granulocytes according to standard methods, so that the remaining population of nucleated cells is enriched for placental stem cells relative to other cell types in placental perfusate. Such enriched populations of cells may be used unfrozen, or may be frozen for later use. If the population of cells is to be frozen, a standard cryopreservative (e.g., DMSO, glycerol, Epilife™ Cell Freezing Medium (Cascade Biologics) is added to the enriched population of cells before it is frozen.

The pharmaceutical compositions described herein may comprise one or more agents that induce cell differentiation. In certain embodiments, an agent that induces differentiation includes, but is not limited to, Ca2+, EGF, α-FGF, β-FGF, PDGF, keratinocyte growth factor (KGF), TGF-β, cytokines (e.g., IL-1α, IL-1β, IFN-γ, TFN), retinoic acid, transferrin, hormones (e.g., androgen, estrogen, insulin, prolactin, triiodothyroxine, hydrocortisone, dexamethasone), sodium butyrate, TPA, DMSO, NMF, DMF, matrix elements (e.g., collagen, laminin, heparan sulfate, Matrigel™), or combinations thereof.

In another embodiment, the pharmaceutical compositions provided herein may comprise one or more agents that suppress cellular differentiation. In certain embodiments, an agent that suppresses differentiation includes, but is not limited to, human Delta-1 and human Serrate-1 polypeptides (see, Sakano et al., U.S. Pat. No. 6,337,387), leukemia inhibitory factor (LIF), stem cell factor, or combinations thereof.

The pharmaceutical compositions provided herein may be treated prior to administration to an individual with a compound that modulates the activity of TNF-α. Such compounds are disclosed in detail in, e.g., U.S. Application Publication No. 2003/0235909, which disclosure is incorporated herein in its entirety. Such compounds are referred to as IMiDs (immunomodulatory compounds) and SELCIDS® (Selective Cytokine Inhibitory Drugs), and particularly compounds available under the trade names ACTIMID™, REVIMID™ and REVLIMID™.

### 6. EXAMPLES

### 6.1 Example 1

### Collection of Umbilical Cord Blood and Placental Stem Cells

This example illustrates the collection of umbilical cord blood and placental stem cells.

### 6.1.1 Collection of Umbilical Cord Blood

Umbilical cord blood is collected using an umbilical cord blood collection kit such as described in U.S. Patent No. 7,147,626, the contents of which are incorporated by reference in their entirety.

Collection kits, containing standard chucks, sterile gauze pad, povidine iodine swabs, sterile alcohol pads, plastic umbilical cord blood clamps, slide clip or hemostat clamps and leak proof resealable bags or canisters are used.

The collection can be performed before the placenta is delivered (in utero collection), after the placenta is delivered (ex utero collection) or during a C-section, prior to delivery of placenta.

Briefly, the venipuncture site on the distal site on the umbilical cord is sterilized. The collection tubing leading from the large collection bag is clamped, the cap is removed from the needle, and the umbilical vein is cannulated with the bevel of the needle facing down toward the umbilical vein. The clamp is removed to allow the blood to flow and collection bag is lowered below the cannulation site to allow the blood to fill the collection bag by gravity.

When the blood flow stops, the venipuncture site is clamped and the needle is withdrawn from the umbilical vein. The collection bag is labelled and put into the insulated shipping container.

The placenta with the clamped umbilical cord blood is placed in the leak proof resealable bag and the bag is then properly sealed and labeled.

After collection, viability of umbilical cord blood cells is determined by hemocytometer after trypan blue staining.

### 6.1.2 Isolation of Placental Stem Cells

Following exsanguination of the umbilical cord and placenta, the placenta was placed in a sterile, insulated container at room temperature and delivered to the laboratory within 4 hours of birth. Placentas were discarded if, on inspection, they had evidence of physical damage such as fragmentation of the organ or avulsion of umbilical vessels. Placentas were maintained at room temperature (23±2° C.) or refrigerated (4° C.) in sterile containers for 2 to 20 hours. Periodically, the placentas were immersed and washed in sterile saline at 25±3° C. to remove any visible surface blood or debris. The umbilical cord was transected approximately 5 cm from its insertion into the placenta and the umbilical vessels were cannulated with TEFLON® polymer or polypropylene catheters connected to a sterile fluid path allowing bi-directional perfusion of the placenta and recovery of the effluent fluid. The system employed enabled all aspects of conditioning, perfusion and effluent collection to be performed under controlled ambient atmospheric conditions as well as real-time monitoring of intravascular pressure and flow rates, core and perfusate temperatures and recovered effluent volumes. A range of conditioning protocols were evaluated over a 24 hour post-partum period and the cellular composition of the effluent fluid was analyzed by flow cytometry, light microscopy and colony forming unit assays.

Placental Conditioning:

The placenta was maintained under varying conditions in an attempt to simulate and sustain a physiologically compatible environment for the proliferation and recruitment of residual cells. The cannula was flushed with IMDM serum-free medium (GibcoBRL, NY) containing 2 U/ml heparin (EJkins-Sinn, NJ). Perfusion of the placenta continued at a rate of 50 mL per minute until approximately 150 mL of perfusate was collected. This volume of perfusate was labeled “early fraction”. Continued perfusion of the placenta at the same rate resulted in the collection of a second fraction of approximately 150 mL and was labeled “late fraction”. During the course of the procedure, the placenta was gently massaged to aid in the perfusion process and assist in the recovery of cellular material. Effluent fluid was collected from the perfusion circuit by both gravity drainage and aspiration through the arterial cannula.

Placentas were obtained from delivery rooms along with cord blood after obtaining written parental consent, and were processed at room temperature within 12 to 24 hours after delivery. Before processing, the membranes were removed and the maternal site washed clean of residual blood. The umbilical vessels were cannulated with catheters made from 20 gauge Butterfly needles use for blood sample collection. Placentas were then perfused with heparinized (2 U/mL) Dulbecco's modified Eagle Medium (H.DMEM) at the rate of 15 mL/minute for 10 minutes and the perfusates were collected from the maternal sites within one hour and the nucleated cells counted. The perfusion and collection procedures were repeated once or twice until the number of recovered nucleated cells fell below 100/mL. The perfusates were pooled and subjected to light centrifugation to remove platelets, debris and de-nucleated cell membranes. The nucleated cells were then isolated by Ficoll-Hypaque density gradient centrifugation and after washing, resuspended in H.DMEM. For isolation of the adherent cells, aliquots of 5-10×106 cells were placed in each of several T-75 flasks and cultured with commercially available Mesenchymal Stem Cell Growth Medium (MSCGM) obtained from BioWhittaker, and placed in a tissue culture incubator (37° C., 5% CO₂). After 10 to 15 days, the non-adherent cells were removed by washing with PBS, which was then replaced by MSCGM. The flasks were examined daily for the presence of various adherent cell types and in particular, for identification and expansion of clusters of fibroblastoid cells.

Cell Recovery and Isolation:

Cells were recovered from the perfusates by centrifugation at 100×g for 15 minutes at room temperature. This procedure served to separate cells from contaminating debris and platelets. The cell pellets were resuspended in IMDM serum-free medium containing 2 U/ml heparin and 2 mM EDTA (GibcoBRL, NY). The total mononuclear cell fraction was isolated using LYMPHOPREP™ (Nycomed Pharma, Oslo, Norway) according to the manufacturer's recommended procedure and the mononuclear cell fraction was resuspended. Cells were counted using a hemocytometer. Viability was evaluated by trypan blue exclusion. Isolation of mesenchymal cells was achieved by differential trypsinization using a solution of 0.05% trypsin with 0.2% EDTA (Sigma). Differential trypsinization was possible because fibroblastoid cells, including placental stem cells, detached from plastic surfaces within about five minutes whereas the other adherent populations required more than 20-30 minutes incubation. The detached fibroblastoid cells were harvested following trypsinization and trypsin neutralization, using Trypsin Neutralyzing Solution (TNS, BioWhitaker). The cells were washed in H.DMEM and resuspended in MSCGM. Flow cytometry was carried out using a Becton-Dickinson FACSCalibur instrument. FITC and PE labeled monoclonal antibodies, including antibodies for CD10, CD 34, CD44, CD45, CD54, CD90, SSEA3, and SSEA4, were purchased from Becton-Dickinson and Caltag laboratories (S. San Francisco, Calif.), or other suppliers and SH2, SH3 and SH4 antibody producing hybridomas were obtained from the American Type Culture Collection, and reactivities of the monoclonal antibodies in their cultured supernatants were detected by FITC or PE labeled F(ab)′2 goat anti-mouse antibodies. Lineage differentiation was carried out using the commercially available induction and maintenance culture media (BioWhittaker), used as per manufacturer's instructions.

Isolation of Placental Stem Cells:

Microscopic examination of adherent cells in the culture flasks revealed morphologically different cell types, including spindle-shaped cells, round cells with large nuclei and numerous perinuclear small vacuoles, and star-shaped cells with several projections, through one of which the cells were attached to the flask. Similar non-stem cells were observed in the culture of bone marrow, cord and peripheral blood; therefore, these cells were considered to be non-stem cell in nature. The fibroblastoid cells, appearing last as clusters, were candidates for being mesenchymal-like stem cells and were isolated by differential trypsinization and subcultured in secondary flasks. Post-trypsinization phase microscopy of the rounded cells revealed the cells to be highly granulated, and similar to bone marrow-derived MSC produced in the laboratory or purchased from commercial sources. When subcultured, the placental stem cells, in contrast to their earlier phase, adhered within hours, assumed a characteristic fibroblastoid shape, and formed a growth pattern identical to the reference bone marrow-derived MSC. Moreover, during subculturing and refeeding, the loosely bound mononuclear cells were washed out and the cultures remained homogeneous and devoid of any visible non-fibroblastoid cell contaminants.

Flow Cytometry:

The expression of placental stem cell surface markers, including CD10, CD29, CD34, CD38, CD44, CD45, CD54, CD90, SSEA3 and SSEA4 was assessed by flow cytometry. Expression of OCT-4 and ABC-p was assessed by RT-PCR using known primers for these markers.

### 6.2 Example 2

### Treatment of Premature Infants with Umbilical Cord Blood and Placental Stem Cells

Six premature infants born at gestational age of between 23 weeks to 36 weeks exhibiting Respiratory Distress Syndrome (RDS) or Acute Respiratory Distress Syndrome (ARDS), anemia, intraventricular hemorrhage, necrotizing enterocolitis, retinopathy of prematurity, chronic lung disease (bronchopulmonary dysplasia), an infection, patent ductus arteriosus, apnea, low blood pressure, hyperbilirubinemia, incomplete development of lung, eye, immune system, brain, heart, liver or kidney are treated with umbilical cord blood and placental stem cells.

Umbilical cord blood is collected as described in Example 1 and placental stem cells are obtained by perfusion or by enzymatic digestion, as described in U.S. Pat. No. 8,057,788, the disclosure of which is hereby incorporated by reference. Umbilical cord blood and placental stem cells are combined at a ratio of 1:1. Umbilical cord blood and placental stem cells are characterized by FACS analysis. Umbilical cord cells and placental stem cells are injected intravenously to the premature infants at dosages of about 1×10⁵ to 1×10⁶ CD34⁺ cells per kilogram body weight of the premature infants one week after delivery and two weeks after delivery.

Prior to and after administration of umbilical cord blood and placental stem cells, blood pressure, heart rate, respiratory rate, and counts of various blood cell types of the premature infants are measured.

### 6.3 Example 3

### Characterization of Cells from Cord Blood and Placental Perfusate

The following experiments were performed to demonstrate the feasibility of producing a combined human placental perfusate (HPP) and umbilical cord blood (UCB) from preterm placenta and to determine the cellular composition of the combined product as compared to cell isolated from term placenta.

Overall results show that (1) It is feasible to collect HPP and UCB from preterm placenta; (2) the number of total nuclear cells (TNC) isolated from preterm placenta is comparable to that isolated from term placenta; (3) The TNC content of HPP/UCB isolated from preterm placenta is significantly higher than that shown in term placenta when normalized to similar weight; (4) the cellular composition of umbilical cord blood cells isolated from preterm placenta is different than that of term placenta; CD38⁺CD45⁺ cells are statistically significantly higher in term placenta compared to preterm placenta (p-value of 0.0146), while CD38⁻CD45⁻ cells are statistically significantly higher in preterm placenta (p-value of 0.0335); and (5) the cellular composition of HPP isolated from preterm placenta is not significantly different than term placenta.

Thus, it is feasible to generate HPP and UCB stem cells from preterm placenta in quantities which substantially exceeds the conventional methods to isolate UCB stem cells. The combined cellular product should be useful to treat complications associated with premature birth since it could be rich source of several progenitor cells with trophic capacity to protect endogenous cell death against hypoxia and differentiation capacity to form blood, angiogenic and neuronal cells in vivo.

### 6.3.1 Methods

Subjects:

Women awaiting elective caesarean sections were recruited in the antenatal clinic and those having spontaneous deliveries were recruited on the delivery suite.

Cord Blood and Placenta Collection:

The placenta was collected, and the umbilical cord was cut and clamped. As much cord blood as possible was drained from the umbilical cord. The cord was then clamped again to prevent further blood loss and the placenta and cord blood were immediately delivered to the lab.

Laboratory Processing:

On arrival in the lab, within 10-15 minutes from the time of delivery), the placenta was weighed and the placental membranes removed. The placenta and cord were assessed to see if they are suitable for perfusion, e.g., the cord is completely attached to the placenta and there is no sign of a tear and also that there are no deep tears in the placenta. The placenta is then covered with the preservation solution. The placenta is then placed in a refrigerator for 48 hours. A sample of cord blood was run immediately for Cell-Dyn and FACS analysis.

Placental Perfusion:

Placentas were perfused and the perfusate cryopreserved. Pressure controlled perfusions were accomplished using a Masterflex L/S 7523 programmable peristaltic pump. An umbilical catheter was inserted into each vein of the placenta umbilical cord and connected to a three-way stopcock. This assembly was then connected to a DPT100 disposable pressure transducer from Utah medical and then to Masterflex L/S 16 peristaltic pump tubing, which was in turn connected to a bag of injectible grade saline. Tubing from a blood collection bag was inserted into the umbilical vein to collect the placental blood. Labview software monitored the pressure of the perfusion, and the Masterflex pump was manually adjusted to the pressure desired. Perfusions were limited to 3 hours with volume and NOC testing completed after each hour of perfusion.

Viability Testing of Placenta Perfusate Cells:

Bags containing HPP were mixed thoroughly, and a small aliquot of the HPP was removed from the bag. The contaminating erythrocytes were then lysed by treating the sample with acetic acid. After erythrocyte cell lysis was complete, each sample was subjected to either Trypan Blue staining or counting by a Cell Dyne 3200. To determine percentage of viable cells, the numbers of intact cells in a microscopic field which exclude the uptake of Trypan Blue were determined in triplicate. The number of viable cells was divided by total cell number multiplied by 100.

Flow Cytometry:

Flow cytometry studies were performed using HPP or UCB or combined cells using the following antibodies:

| | | | | | FL5 PE-CY7 | |
|---|---|---|---|---|---|---|
| | FL-FITC | | | FL4 APC | APC-CY7 | FL6 |
| | or | | | or | or APC | Pacific |
| Tube | Alexa488 | FL2-PE | FL3 PerCP | Alexa647 | Alexa750 | Blue |
| 1 | PS | 235a | 7AAD | 38 | 34 | 45 |
| 2 | 90 | 133 | 69 | 38 | 34 | 45 |
| 3 | 44 | 117 | 7AAD | 105 | 34 | 45 |

Cells were washed with buffer, and then re-suspended at a set concentration range (i.e. 1×10⁶ live cells per 100 μL) in buffer. Cells were then stained with fluorescence-conjugated antibodies, incubated, and then washed with buffer to remove excess antibodies. Stained cells were tested on a flow cytometer to determine positive or negative expression of the markers of interest.

### 6.3.2 Results

Pre-term and term placentas were found to have significantly different weights (average of 345 g and 731 g, respectively; p=0.0001). The viability of cells from pre-term and term placentas was not found to differ significantly (viability of 93.13% and 90.84%, respectively; p=0.0724). However, the total nucleated cord blood cells obtainable from pre-term did differ significantly (p=0.0001). A mean of 5.277×10⁷ cells were obtained from pre-term placentas, and a mean of 1.9498×10⁸ cells were obtained from term placentas. Similarly, the total nucleated perfusate cells obtainable from pre-term did differ significantly (p=0.0016). A mean of 1.28×10⁷ TNC were obtained from pre-term placentas, and a mean of 3.76×10⁷ TNC were obtained from term placentas. Thus, the number of nucleated cells obtainable per gram of placental tissue was significantly greater for pre-term placentas than term placentas (p=0.0001). The total perfusate+cord blood nucleated cells that can be obtained from a pre-term placenta is 1.79×10⁸. This is extremely significantly different compared with a term placenta of 5.71×10⁸ for term placenta (p=0.0001). No significant difference in the number of combined total nucleated cells per gram of placental tissue was found.

### 6.3.3 Flow Cytometry

No statistically significant difference between the percentage of CD34⁺ cells, CD38⁻CD45⁺ cells, or CD38⁺CD45⁻ cells in the cord blood from pre-term and term placentas was found. Significantly higher numbers of CD38⁺CD45⁺ cells were found in term placenta (16.68% of TNC vs. 2.0% TNC in pre-term), and significantly higher numbers of CD38⁻CD45⁻ cells are found in pre-term placenta (56.52% vs. 24.58% for term placenta). No statistically significant difference in the percentage of perfusate-derived CD38⁻CD45⁺ cells, CD38⁺CD45⁻ cells, CD38⁺CD45⁺ cells or CD38⁻CD45⁻ cells was found between pre-term and term placentas.

### 6.4 Example 4

### Treatment of Neonatal Brain Injury

This example demonstrates that placental stem cells can be used in the treatment of neonatal brain injury.

Focal cerebral ischemia (FCI), a validated model neonatal brain injury, was induced in post-natal day 7 (P7) Sprague-Dawley rats by occlusion of the middle cerebral artery (see, e.g., Wen et al., 2004, Brain Res. Protoc. 2:76-83) of the rats (“FCI group”). A separate group of rats were exposed to the same FCI dissection procedure as the rats in the FCI group, without occlusion of the middle cerebral artery (“Sham group”).

One hour following surgery, either CD34⁻, CD10⁺, CD105⁺, CD200⁺ placental stem cells or a vehicle control (DMEM) were administered by intracerebroventricular (ICV) injection to the left lateral ventricle of rats in both the FCI group and Sham group. Placental stem cells were administered at two different doses: 4×10⁶ cells/ml and 20×10⁶ cells/ml.

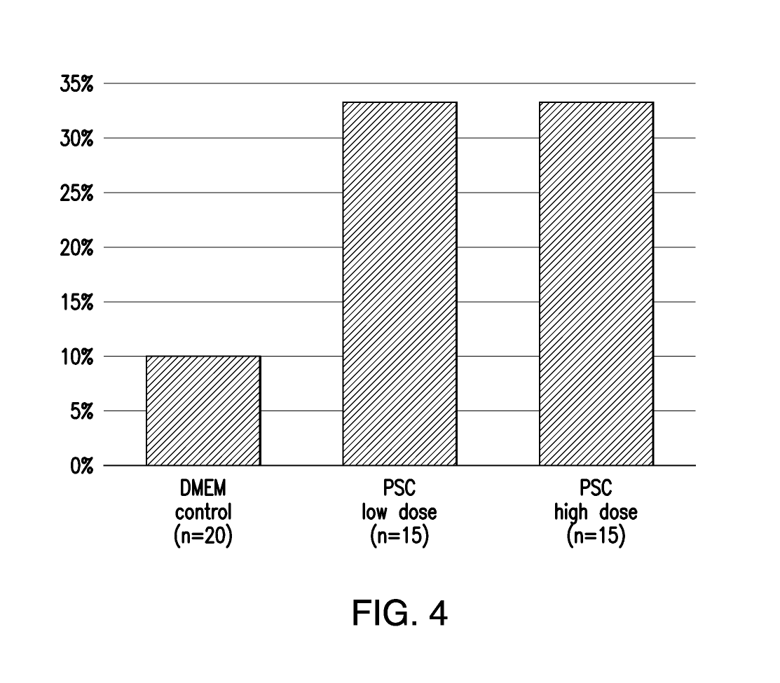

At 24-hours post-surgery, infarct size was comparable across rats in the FCI group, irrespective of whether the rats were administered placental stem cells (at either dose) or vehicle control. However, three weeks following surgery, rats in the FCI group that were treated with placental stem cells (n=15 for both cell doses) demonstrated increased survival as compared to rats treated with vehicle control (n=16) (FIG. 4). Survival rate of placental stem cell-treated rats in the FCI group was the same for both doses of cells administered (FIG. 4). As expected, all rats in the Sham group (i.e., rats administered placental stem cells (n=9) and rats administered vehicle control (n=8)) were alive three weeks after surgery.

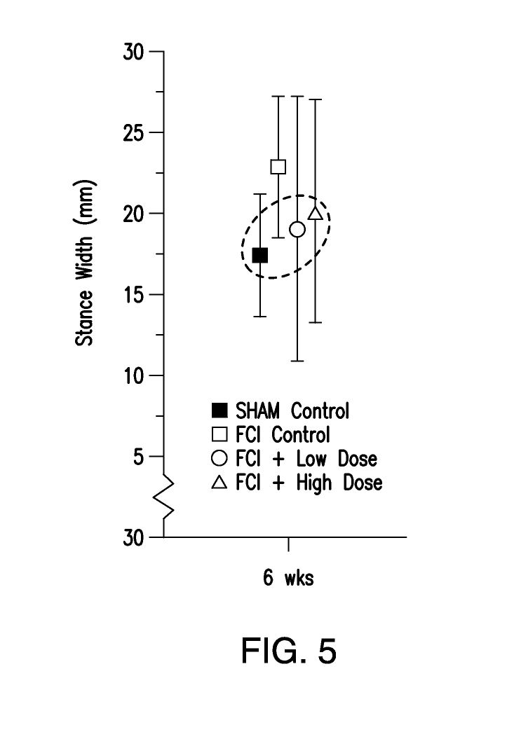

Induction of FCI in rats can cause gait impairment. Gait analyses conducted at six weeks following surgery demonstrated that rats in the FCI group treated with placental stem cells at either dose had improved gait (with values similar to those of rats in the Sham group; see dotted circle of FIG. 5) compared to rats in the FCI group treated with vehicle control, as evidenced by decreased stance width in the placental stem cell treated rats (FIG. 5).

Immunohistochemistry was performed on the brains of rats in the FCI group six weeks following surgery to determine whether administration of placental stem cells had an effect on the neuronal architecture at the infarction site. Placental stem cell-treated rats demonstrated improved neuronal architecture in the post-infarct region compared to rats tin the FCI group that received vehicle control, as evidenced by staining for glial fibrillary acidic protein (GFAP) and microtubule-associated protein 2 (MAP2).

In summary, placental stem cells can be used to improve brain injury in neonatal rats as evidenced by increased survival, improved gait, and improved neuronal architecture.

### EQUIVALENTS

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

1. A method of treating a disorder or condition of an infant, comprising administering to said infant total nucleated cells obtained from placental perfusate or placental stem cells.

2. The method of claim 1, further comprising administering umbilical cord blood or total nucleated cells obtained from umbilical cord blood to the infant.

3. The method of claim 1, wherein said infant is a premature infant.

4. The method of claim 1, wherein said infant is a full-term infant.

5. The method of claim 3, wherein said infant is a neonatal infant.

6. The method of claim 1, wherein said disorder or condition is a neurological disorder or condition, a respiratory disorder or condition, or a gastrointestinal disorder or condition.

7. The method of claim 6, wherein said neurological disorder or condition is caused by ischemia.

8. The method of claim 1, wherein said disorder or condition is selected from the group consisting of Respiratory Distress Syndrome (RDS), Acute Respiratory Distress Syndrome (ARDS), anemia, a neurological deficiency, intraventricular hemorrhage, necrotizing enterocolitis, retinopathy of prematurity, chronic lung disease (bronchopulmonary dysplasia), an infection, patent ductus arteriosus, apnea, low blood pressure, and hyperbilirubinemia.

9. The method of claim 1, wherein said disorder or condition is caused by incomplete development of an organ.

10. The method of claim 1, wherein said placental perfusate or placental stem cells are autologous to the infant.

11. The method of claim 2, wherein said umbilical cord blood is autologous to the infant.

12. The method of claim 1, wherein said placental stem cells are stem cells isolated from placental perfusate.

13. The method of claim 1, wherein said placental stem cells are stem cells contained within placental perfusate.

14. The method of claim 1, wherein said placental stem cells comprise:
a. CD34⁻, CD10⁺, CD105⁺, CD200⁺ stem cells;
b. CD34⁻ stem cells;
c. OCT-4⁺ stem cells;
d. CD200⁺ and HLA-G⁺ stem cells;
e. CD73⁺, CD105⁺, and CD200⁺ stem cells;
f. CD200⁺ and OCT-4⁺ stem cells;
g. CD73⁺ and CD105⁺ stem cells and facilitate the formation of one or more embryoid-like bodies in a population of placental cells comprising said stem cell when said population is cultured under conditions that allow the formation of an embryoid-like body; or
h. OCT-4⁺ stem cells and facilitate the formation of one or more embryoid-like bodies in a population of placental cells comprising the stem cell when said population is cultured under conditions that allow formation of embryoid-like bodies; or any combination thereof.

15. The method of claim 1, wherein total nucleated cells obtained from placental perfusate comprise:
a. CD34⁺CD45⁻ cells;
b. CD34⁺CD45⁺ cells;
c. CD105⁺CD200⁺ cells; or
d. CD34⁺CD45⁺CD41⁺CD61⁺ cells.

16. The method of claim 1, wherein said cells comprise CD34⁺ cells.

17. The method of claim 1, wherein said administering is performed once after birth of the premature infant.

18. The method of claim 1, wherein said administering is performed a plurality of times after birth of the premature infant.

19. The method of claim 1, wherein said administering is performed within one hour, 12 hours, 24 hours, or one week after birth of the premature infant.

20. The method of claim 1, wherein said administration is performed intravenously, subcutaneously, locally, systemically, intraperitoneally, parenterally, or intracerebroventricularly.
